(19) United States
(12) Patent Application Publication
(10) Pub. No.: US 2025/0367348 A1
(43) Pub. Date: Dec. 4, 2025

(54) 3D PRINTING OF 3-LAYERED RETINA AND CHOROID TISSUE

(71) Applicants: Ramot at Tel-Aviv University Ltd., Tel-Aviv (IL); Ichilov Tech Ltd., Tel-Aviv (IL)

(72) Inventors: Tal DVIR, Tel Aviv (IL); Yahel SHECHTER, Tel Aviv (IL); Eric SILBERMAN, Tel Aviv (IL); Aya BARZELAY WOLLMAN, Tel-Aviv (IL); Adiel BARAK, Tel-Aviv (IL)

(73) Assignees: Ramot at Tel-Aviv University Ltd., Tel-Aviv (IL); Ichilov Tech Ltd., Tel-Aviv (IL)

(21) Appl. No.: 19/303,562

(22) Filed: Aug. 19, 2025

Related U.S. Application Data
(63) Continuation of application No. PCT/IL2024/050152, filed on Feb. 8, 2024.
(60) Provisional application No. 63/446,868, filed on Feb. 19, 2023.

(51) Int. Cl. A61L 27/38; A61L 27/52; B33Y 40/20; B33Y 80/00; C12N 5/079; C12N 5/0793

(52) U.S. Cl. CPC A61L 27/3808; A61L 27/3813; A61L 27/52; B33Y 40/20; B33Y 80/00; C12N 5/062; C12N 5/0621; A61L 2430/16; C12N 2533/30; C12N 2533/90; C12N 2537/10

(57) ABSTRACT

A method for reinforcing a cellularized retinal construct fabricated from (i) endothelial cells; (ii) retinal pigment epithelial cells and/or photoreceptors; and (iii) an extracellular matrix (ECM) hydrogel is disclosed. The method comprises contacting the construct with a biocompatible small-molecule reinforcing agent that is capable of chemically interacting with the ECM hydrogel under conditions that maintain viability of the cells, to thereby increase a compressive modulus of the ECM hydrogel by at least 10%.

### RELATED APPLICATIONS

This application is a Continuation of PCT Patent Application No. PCT/IL2024/050152 having International filing date of Feb. 8, 2024, which claims the benefit of priority under 35 USC § 119 (e) of U.S. Provisional Patent Application No. 63/446,868 filed on Feb. 19, 2023. The contents of the above applications are all incorporated by reference as if fully set forth herein in their entirety.

### FIELD AND BACKGROUND OF THE INVENTION

The present invention, in some embodiments thereof, relates to engineering of cellularized retinal constructs, and more particularly, but not exclusively, to small molecules capable of reinforcing same.

Age-related macular degeneration (AMD) is a chronic disease of the central retina (macula) that is the leading cause of blindness in Western countries. Late stages of AMD are characterized by either choroidal neo-vascularization or by geographic atrophy (GA). GA is characterized as a sharply defined area in the macula in which there is atrophy of the choriocapillaris, retinal pigment epithelium, and photoreceptors. Currently, there is no available cure for GA. Recently the FDA approved two complement inhibitor drugs to treat GA, however these therapies are only able to slow down the progression of the disease to some extent and are not curative³. In the healthy retina, the layers are organized in a hierarchical pattern, in which each layer is cardinal for the function and survival of the next. The choriocapillaris are the closest to Bruch's membrane and the pigmented epithelial layer; they supply oxygen and nutrients to the outer retina. Next, the retinal pigmented epithelial cells (RPE) provide the cardinal metabolic support to the photoreceptor cells on top. In AMD, as well as in other maculopathies this symbiotic relationship and structure of the choriocapillaris/RPE/photoreceptors is lost.

Tissue engineering involves the design and creation of functional living tissues and organs from cells and biomaterials using engineering principles. Throughout the years, researchers have developed various fabrication technologies and approaches that have the potential to transform the field of medicine, including 3D printing, electrospinning, and molded scaffolds. Aiming to restore vision loss due to RPE degradation, in recent years, tissue engineering approaches such as RPE and photoreceptor cell injection or transplantation of pre-engineered retinal tissue parts showed promising results^{9,10,11}. In cell injection, purified photoreceptors or RPE or progenitor cells^{12,13} were injected into a wide area in the retina and could directly contact host cells^{14,15}. However, in such cases, the cells could not form a structured layer that may assist in maturation^{14,15}. Contrary, transplantation of RPE cell sheets allowed the delivery of a structured mature layer of the RPE, which could better survive and properly interact with the host tissue¹⁶. For example, a groundbreaking clinical trial using autologous iPSC derived RPE for the treatment for dry age-related macular degeneration is currently ongoing¹⁷. However, since AMD is typically diagnosed at a late stage, when patients already suffer from distorted vision or central visual field defects due to photoreceptor loss¹⁸, replacing the RPE layer alone can only support remaining photoreceptors and cannot restore lost vision. Furthermore, in advanced cases where the atrophy includes degeneration of the choriocapillaris, it is essential to engineer a triple-layer tissue, which includes the choriocapillaris, RPE and photoreceptors. To note, the co-culture of RPE and photoreceptor cells is challenging, as these cells require different molecules for the initial cell assembly. It is important to ensure that the different cell types in the co-culture system maintain their distinct identities and functions. This may involve using cell-specific culture media and supplements to support the growth and differentiation of each cell type. For example, endothelial cells require factors such as vascular endothelial growth factor (VEGF), basic fibroblast growth factor (bFGF), epidermal growth factor (EGF), and platelet-derived growth factor (PDGF). However, RPE cells also require insulin-like growth factor 1 (IGF-1), and photoreceptors need brain-derived growth factor and ciliary neurotrophic factor for their growth.

Personalized ECM-based hydrogel as a bio-ink for advanced 3D printing techniques is known²⁰⁻²³. The combination of the hydrogel and the patient's own cells was used to print thick, vascularized, and perfusable patches that fully matched the immunological, biochemical, and anatomical properties of the patient. Moreover, the technology was used for the 3D printing of volumetric structures such as a small-scale human heart²⁰.

International Patent Application No. WO2009/085547 teaches the generation of decellularized omentum scaffolds for tissue engineering. International Patent Application No. WO2009/085547 does not teach use of the decellularized omentum scaffolds for cardiac engineering.

International Patent Application No. WO2014/207744 teaches the generation of decellularized omentum scaffolds for tissue engineering. International Patent Application No. WO2014/207744 does not teach conditions for decellularizing human omentum.

U.S. Patent Publication No. 20050013870 teaches a scaffold comprising decellularized extracellular matrix of a number of body tissues including omentum. The body tissues have been conditioned to produce a biological material such as a growth factor.

Porzionato et al. (Italian Journal of Anatomy and Embryology, Volume 116, 2011 and Eur J Histochem. 2013 Jan. 24;57 (1): e4. doi: 10.4081/ejh.2013.e4) teaches decellularized omentum.

Additional background art includes Gilbert et al., Biomaterials 27 (2006) 3675-3683 and Flynn et al., Biomaterials 31 (2010), 4715-4724.

U.S. Patent Publication No. 2009/0163990 and 2020/0101198-A1 teaches methods of decellularizing omentum.

Soluble forms of decellularized extracellular matrix are known in the art as described in Acta Biomaterialia, Volume 9, Issue 8, August 2013, Pages 7865-7873 and Singelyn et al., J Am Coll Cardiol. Feb. 21, 2012; 59 (8): 751-763.

Additional background art includes Masaeli et al., Biofabrication 12 (2020) 025006 and Song et al., Nature Methods, https://doi (dot) org/10.1038/s41592-022-01701-1 and WO2019/234738.

### SUMMARY OF THE INVENTION

According to aspects of the invention, there is provided a method for reinforcing a cellularized retinal construct fabricated from:
(i) endothelial cells;
(ii) retinal pigment epithelial (RPE) cells and/or photoreceptors; and
(iii) an extracellular matrix (ECM) hydrogel,
the method comprising:
contacting the cellularized retinal construct with a biocompatible small-molecule reinforcing agent that is capable of chemically interacting with the ECM hydrogel under conditions that maintain viability of the cells, to thereby increase a compressive modulus of the ECM hydrogel by at least 10%.

In some embodiments, the chemically interacting effects cross-linking of the ECM hydrogel.

In some embodiments, the reinforcing agent is capable of chemically interacting with the ECM hydrogel via a Click reaction.

In some embodiments, the Click reaction forms a Schiff base (an imine bond).

In some embodiments, the reinforcing agent is a polyaldehyde.

In some embodiments, the reinforcing agent is an oxidized, poly-aldehyde saccharide.

In some embodiments, the contacting is with a culturing medium that comprises the reinforcing agent.

In some embodiments, the reinforcing agent is an oxidized, poly-aldehyde saccharide and wherein a concentration of the reinforcing agent in the medium is less than 0.1% by weight.

In some embodiments, the conditions comprise incubation at 37° C.

In some embodiments, the method further comprises generating the cellularized retinal construct prior to the contacting by sequentially forming a plurality of layers on a receiving medium, wherein a first of the layers comprises the endothelial cells and a second of the layers comprises the RPE cells or the photoreceptors.

In some embodiments, the second layer comprises RPE cells and a third layer comprises the photoreceptors.

In some embodiments, the RPE cells express at least one marker selected from the group consisting of ZO1, OTX½, PAX6, BEST1 and RPE65 prior to the contacting.

In some embodiments, the RPE cells comprise a cobblestone morphology prior to the contacting.

In some embodiments, the photoreceptors express nestin prior to the contacting.

In some embodiments, the method further comprises culturing the cellularized retinal construct for at least 3 days following the generating and prior to the contacting.

In some embodiments, the generating the first layer comprises 3D printing at least one tubular structure from a bioink comprising the endothelial cells and the ECM hydrogel.

In some embodiments, the bioink further comprises a support medium comprising calcium alginate hydrogel particles.

In some embodiments, the internal diameter of the at least one tubular structure is between 200-500 microns.

In some embodiments, the method further comprises dissolving the support medium prior to forming the second layer.

In some embodiments, the ECM hydrogel is generated from decellularized omentum.

In some embodiments, the RPE cells and/or the endothelial cells are generated ex vivo from pluripotent stem cells.

In some embodiments, the pluripotent stem cells comprise induced pluripotent stem cells (iPSCs).

According to aspects of the invention, there is provided a of generating an engineered cellularized retinal construct comprising:
(a) culturing endothelial cells in the presence of an ECM hydrogel derived from decellularized omental tissue to generate a layer of endothelial cells;
(b) culturing RPE cells on the layer of endothelial cells to generate a layer of RPE cells; and
(c) culturing photoreceptor cells on the layer of RPE cells, thereby generating the cellularized retinal construct.

In some embodiments, the method further comprises contacting the retinal construct with a biocompatible small-molecule reinforcing agent that is capable of chemically interacting with the ECM hydrogel under conditions that maintain viability of the cells, to thereby increase a compressive modulus of the ECM hydrogel by at least 10%.

In some embodiments, the chemically interacting effects cross-linking of the ECM hydrogel.

In some embodiments, the reinforcing agent is capable of chemically interacting with the ECM hydrogel via a Click reaction.

In some embodiments, the Click reaction forms a Schiff base (an imine bond).

In some embodiments, the reinforcing agent is a polyaldehyde.

In some embodiments, the reinforcing agent is an oxidized, poly-aldehyde saccharide.

In some embodiments, the contacting is with a culturing medium that comprises the reinforcing agent.

In some embodiments, the reinforcing agent is an oxidized, poly-aldehyde saccharide and wherein a concentration of the reinforcing agent in the medium is less than 0.1% by weight.

In some embodiments, the conditions comprise incubation at 37° C.

In some embodiments, the RPE cells express at least one marker selected from the group consisting of ZO1, OTX½, PAX6, BEST1 and RPE65 prior to step (c).

In some embodiments, the RPE cells comprise a cobblestone morphology prior to step (c).

In some embodiments, the method further comprises culturing the cellularized retinal construct for at least 3 days following step (c) and prior to the contacting the retinal construct with the biocompatible small-molecule reinforcing agent.

In some embodiments, the layer of endothelial cells is generated by 3D printing at least one tubular structure from a bioink comprising the endothelial cells and the ECM hydrogel.

In some embodiments, the bioink further comprises a support medium comprising calcium alginate hydrogel particles.

In some embodiments, the internal diameter of the tubular structure is between 200-500 microns.

In some embodiments, the method further comprises dissolving the support medium prior to forming the layer of RPE cells.

According to aspects of the invention, there is provided a cellularized, engineered retinal construct generated according to the methods described herein.

According to aspects of the invention, there is provided a cellularized engineered retinal construct comprising endothelial cells and retinal cells distributed within a chemically cross-linked ECM hydrogel, wherein the ECM hydrogel is chemically cross-linked by a biocompatible small-molecule reinforcing agent that is capable of chemically interacting with the ECM hydrogel under conditions that maintain viability of the cells, and wherein a compressive modulus of the ECM hydrogel is higher by at least 50% than a compressive modulus of the ECM hydrogel which is not chemically cross-linked,

According to aspects of the invention, there is provided a method of treating a disease or condition associated with a damaged retina in a subject in need thereof, the method comprising implanting the cellularized construct described herein into the subject, thereby treating the condition.

In some embodiments, the implanting the cellularized construct is effected at the subretinal space of the eye.

In some embodiments, the disease or condition is selected from at least one of retinitis pigmentosa, lebers congenital amaurosis, hereditary or acquired macular degeneration, age related macular degeneration (AMD), Best disease, retinal detachment, gyrate atrophy, choroideremia, pattern dystrophy, RPE dystrophies, Stargardt disease, RPE and retinal damage due to damage caused by any one of photic, laser, inflammatory, infectious, radiation, neovascular or traumatic injury.

According to aspects of the invention, there is provided a cellularized construct for use in treating a disease or condition associated with a damaged retina.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1 illustrates the schematic of the fabrication process according to embodiments of the invention. Blood vessel network was 3D printed. After initial cell assembly, RPE cells were deposited on top of the printed structure. Following 7-day cultivation for RPE monolayer assembly, photoreceptors were deposited to interact with the apical membrane of the RPE.

FIGS. 2A-F illustrate fabrication of the ECM-based hydrogel. A) Native porcine omentum. B) Decellularized omentum. C) ECM hydrogel. The ECM formed a weak gel at room temperature (left) and became a viscous, crosslinked hydrogel at 37° C. (right). D, E) Rheological measurements of 1% ECM hydrogel. D) Post-gelation frequency sweep, E) Time sweep at 37° C. F) SEM image of the hydrogel. Scale bar=5 μm.

FIGS. 3A-F illustrate engineering the choroid layer. A) The printed structure. B) GFP-HUVECs form a vessel-like structure in culture. C) Printed vessel during perfusion. D) Two blood vessels printed in parallel. Scale bar=500 μm. E) The endothelial cells migrated out of the lumen to form a capillary bed. Scale bar=100 μm. F) Imaging of RFP-HUVECs printed in a vessel-like structure to form an open lumen (day 14). Scale bar=100 μm.

FIGS. 4A-N illustrate engineering the RPE layer on top of the printed vascular layer. A) RPE cells in co-culture with endothelial cells expressed ZO1 (green). F-actin appears in orange and nuclei in blue. Scale bar=50 μm; B-E) RPE specific markers B) OTX½ (green) Scale bar=50 μm; C) PAX6 (green). Scale bar=50 μm; D) BEST1 (pink) Scale bar=10 μm; E) RPE65 (pink) Scale bar=50 μm. F) A cross-section view of the blood vessel-RPE layer construct (day 28). Scale bar=150 μm. G) Polarity of the cells was demonstrated by the location of BEST1 (pink) within the RPE layer Scale bar=5 μm. H) Phagocytosis of latex fluorescent beads by the RPE cells after 20 days of culture of the blood vessel-RPE layer construct. Scale bar=10 μm. I) Calcium change in response to ATP addition to the blood vessel-RPE construct (presented as a change from the baseline fluorescence; F/FO). J) TEER measurements across the RPE layer. K) hES-RPE cells in co-culture with hiPSC-EC show defined cell borders and an overall ‘cobblestone’ appearance. Scale bar=10 μm. L) A cross-section view of the RPE layer, polarity of the cells is demonstrated by the location of ezrin (green) on the apical side and beta-catenin (orange) on the basal side of the monolayer. Scale bar=10 μm. M) hES-RPE cells in co-culture with endothelial cells expressed the RPE-specific markers PAX6 (green), BEST1 (red) N) OTX½ (green), and RPE65 (red). Scale bar=10 μm.

FIGS. 5A-F illustrate fabrication of the 3-layer retina-like structure. A) On day 27, RPE cells expressing BEST1 (yellow) in co-culture with endothelial cells and photoreceptors expressing nestin (pink). Scale bar=50 μm. B) A cross-section view of the RPE-photoreceptors construct, the RPE expressed ZO1 (green) underneath photoreceptors cells that expressed nestin (pink). Scale bar=10 μm. C) TEM images of ARPE-19 cells after 104 days in the tri-culture of HUVECs and 661w cells display pigmentation (black arrow), microvilli (blue arrow), and desmosomes (green arrow). Left image scale bar=5 μm, middle image scale bar=1 μm, right image scale bar=200 nm. D-F) Immunostaining for Bruch's membrane proteins, D) Collagen I (pink) and Collagen IV (green). The cells appear in yellow (F-actin), nuclei in blue. Scale bar=50 μm. E) Non-cellular Control of Collagen I and IV. F) Laminin staining. The cells appear in yellow (F-actin), nuclei in blue. Scale bar=10 μm.

FIG. 6 presents the chemical structures of an exemplary reinforcing agent according to some of the present embodiments.

FIGS. 7A-K presents the reinforcing effect of an exemplary reinforcing agent of on an exemplary ECM-based hydrogel. FIG. 7A presents a schematic illustration of a reinforcing process according to some embodiments of the present invention. Tissues are fabricated using 3D printing technology, which deposits cells and ECM fibers with random orientation (I); Cells are then allowed to self-organize and mature in a soft gel (II); and the engineered tissue is thereafter thoroughly reinforced by the diffusion of SOx into the tissue (III). FIG. 7B presents a schematic illustration of the reaction of the poly-aldehyde SOx with amine moieties present in collagen via “Click” chemistry to form imine bridges. FIG. 7C is a bar graph showing the viability of primary umbilical vein endothelial cells (HUVECs) grown in 2D culture containing various concentration of SOx for 48 hours. FIG. 7D presents comparative plots showing the shear thinning behavior of a non-reinforced hydrogel, and hydrogel reinforced in the present of 0.03 or 0.07% Sox, at 37° C. FIG. 7E is a bar graph showing the hydrogel bulk modulus with and without exposure to SOx. FIG. 7F is a bar graph showing the absorbance of a solution of ninhydrin that was allowed to react with the hydrogel, normalized to the control's value, indicating the portion of the amine groups in the hydrogel that reacted with SOx. FIGS. 7G and 7H present images showing the fiber morphology of ECM-based hydrogel before (FIG. 7G) and after (FIG. 7H) exposure to SOx. FIG. 7I is a bar graph showing the average pore size as calculated for the images shown in FIGS. 7G and 7H. FIG. 7I presents comparative plots showing the % of gel that remained after incubation in the presence of the SDS disintegrating detergent overnight. FIG. 7K presents comparative plots showing the % of the gel that remained after incubation in the presence of collagenase for 2 weeks.

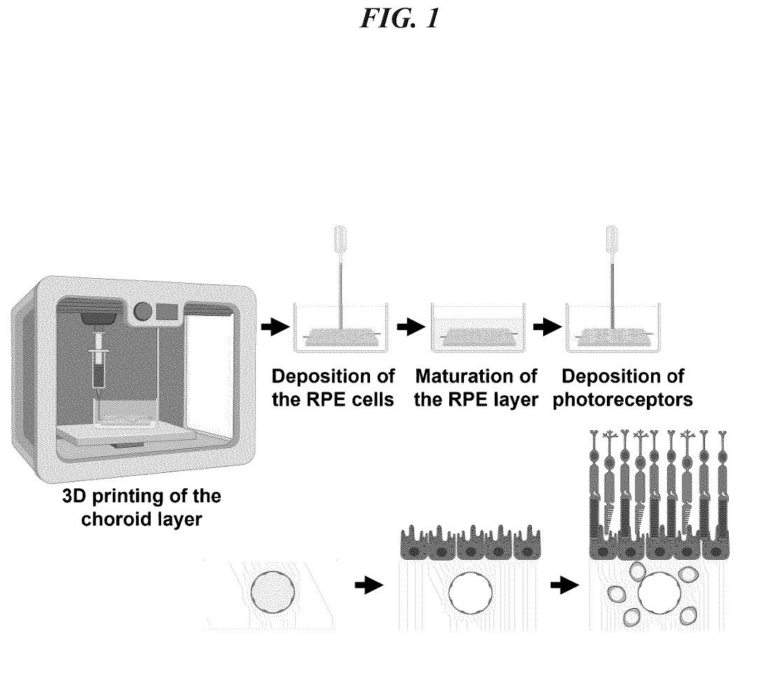

### DESCRIPTION OF SPECIFIC EMBODIMENTS OF THE INVENTION

The present invention, in some embodiments thereof, relates to engineering of cellularized retinal constructs, and more particularly, but not exclusively, to small molecules capable of reinforcing same.

The principles and operation of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

When the complex of choriocapillaris/RPE/photoreceptors malfunctions, severe retinal degeneration can ultimately result in irreversible vision loss. Currently, treatment methods only address exudative AMD leaving 90% of AMD patients with nonexudative, and similar maculopathies with central atrophy with no curative treatment. As biofabrication technologies advance, complex cellular and tissue structures with high physiological relevance can be engineered.

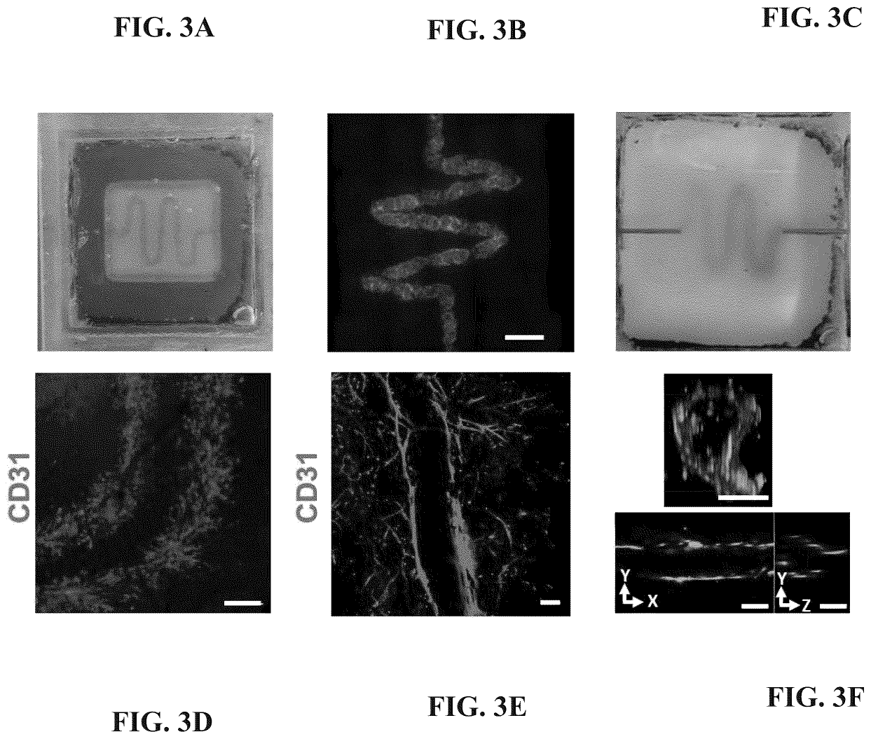

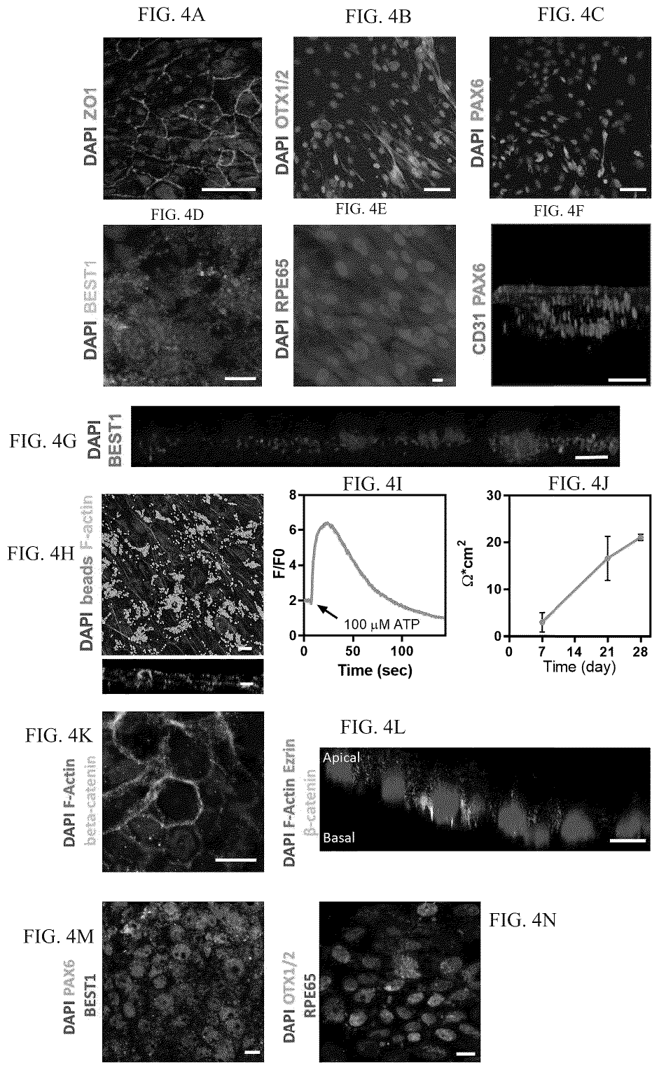

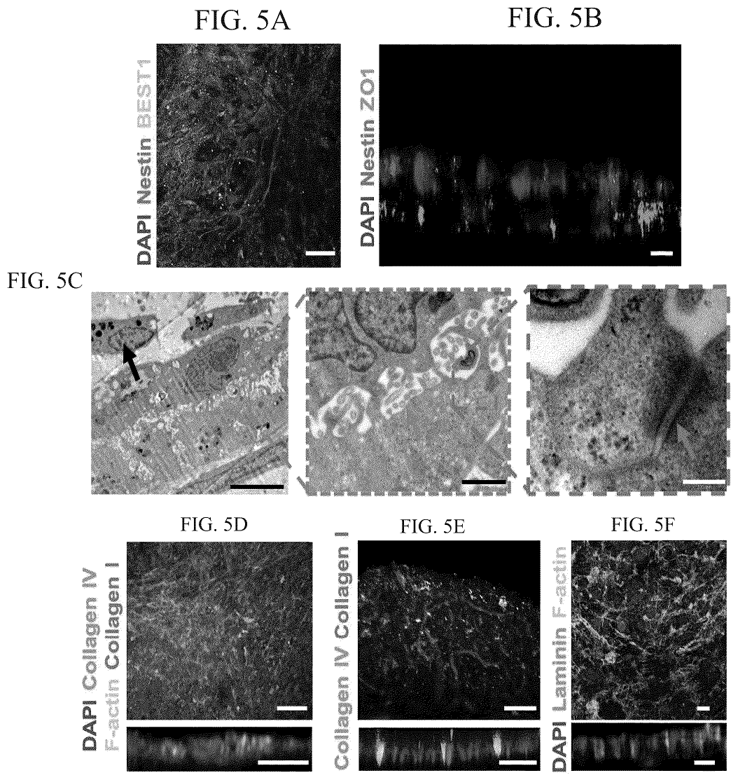

The present inventors have now developed a process for fabricating a 3-layer retina-like structure. As a proof of concept, the fabricated structure was composed of human endothelial cells, RPE and photoreceptor cell lines, and an ECM-based hydrogel. As the three cell types require different cultivation conditions for their initial assembly and maturation, a step-by-step tissue fabrication was performed. Initially, a blood vessel network was 3D printed (FIGS. 3A-F). Subsequently, RPE cells were deposited on top of the vasculature layer (FIGS. 4A-N). The cells were allowed to assemble into a polarized monolayer, and only then, the photoreceptor cells were added to interact with the apical membrane of the RPE layer. The entire structure was further cultured for maturation (FIGS. 5A-F).

Whilst further reducing the present invention to practice, the present inventors conceived of adding a small molecule in order to reinforce the construct following deposition of the photoreceptor cells.

Oxidized sucrose (SOx) was chosen as a non-limiting small molecule in order to reinforce the pre-printed retinal construct. It was hypothesized that adding SOx to the tissue growth medium as a post-printing and post-tissue assembly reinforcer would give the cells time to form cell-cell and cell-matrix interactions within the soft hydrogel and form a tissue.

The present inventors found that the retinal construct was more stable and rigid in the presence of the reinforcing molecule and much easier to handle. The construct could be cultured for over two weeks before the reinforcing molecule was added to the growth media as a final step prior to implantation.

The present inventors conceive that such retina-like structure may be able to attenuate the deterioration of the damaged retina and, in theory, due to the existence of the photoreceptors, also to regenerate it.

Thus, according to an aspect of the invention there is provided a method for reinforcing a cellularized retinal construct fabricated from:
(i) endothelial cells;
(ii) retinal pigment epithelial cells and/or photoreceptors; and
(iii) an extracellular matrix (ECM) hydrogel,
the method comprising:
contacting the cellularized retinal construct with a biocompatible small-molecule reinforcing agent that is capable of chemically interacting with the ECM hydrogel under conditions that maintain viability of the cells, to thereby increase a compressive modulus of the ECM hydrogel by at least 10%.

According to some of any of the embodiments described herein, the method is such that a compressive modulus of the ECM-based hydrogel upon contacting the cellularized engineered construct is higher by at least 10%, or at least 20%, or by at least 30%, or by at least 50%, or by at least 60%, or by at least 70%, or by at least 80%, or by at least 90%, or by at least 100%, compared to the compressive modulus of the hydrogel-based hydrogel before contacting the reinforcing agent, and/or compared to the same hydrogel-based hydrogel when the cellularized engineered construct is generated without contacting the reinforcing agent.

According to some embodiments, a compressive modulus of the ECM-based hydrogel is higher by at least 10%, or at least 20%, or by at least 30%, or by at least 50%, or by at least 60%, or by at least 70%, or by at least 80%, or by at least 90%, or by at least 100% than a compressive modulus of the same ECM-based hydrogel which is not chemically cross-linked, and which can be physically cross-linked as a result of the physically cross-linked fibrous network that provides the hydrogel.

Herein and in the art, the phrase “compressive modulus”, which is also referred to in the art compressive elastic modulus or compressive modulus of elasticity, describes a mechanical property that reflects the ability of a material to resist deformation under compressive loading, and accordingly is a measure of the material's stiffness in compression. When a material is subjected to a compressive force, it undergoes deformation or compression. The compressive modulus quantifies how much the material will deform under this compressive stress, is expressed in units of pressure. According to some embodiments, the compressive modulus is determined as described in the Examples section below.

The term “cellularized retinal construct” (also referred to as a tissue) refers to a three-dimensional cellular aggregate comprising at least one type of specialized cell of the retina. Typically, at least a portion of the cells interact with one another and perform at least one retinal function.

According to a particular embodiment, the cells are intact (i.e., whole), and preferably viable.

The cells may be primary cells, immortalized cells or derived from cell lines.

The cells may be fresh, frozen or preserved in any other way known in the art (e.g., cryopreserved).

In one embodiment, the cells used to fabricate the construct are genetically modified (e.g. to express a therapeutic agent or a detectable moiety) by any suitable method known in the art.

The cellularized construct may comprise one of more layers of cells. The layer may be a monolayer of a multi-layer. Each layer may comprise a single cell type or multiple cell types.

The cells of the cellularized construct may be derived from any mammalian organism including for example (e.g., human, porcine, rabbit, rodent, bovine). According to a particular embodiment, the cells are human cells.

According to a particular embodiment the cells are derived from (or comprise) stem cells—e.g., adult stem cells such as mesenchymal stem cells or pluripotent stem cells such as embryonic stem cells or induced pluripotent stem cells (iPSCs). The stem cells may be modified so as to undergo ex vivo differentiation prior to engineering of the construct or may be used as pluripotent stem cells and further differentiated in situ prior to implantation.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, preferably before 10 weeks of gestation, and cells originating from an unfertilized ova which are stimulated by parthenogenesis (parthenotes).

It will be appreciated that commercially available stem cells can also be used according to some embodiments of the invention. Human ES cells can be purchased from the NIH human embryonic stem cells registry [Hypertext Transfer Protocol://grants (dot) nih (dot) gov/stem_cells/registry/current (dot) htm]. Non-limiting examples of commercially available embryonic stem cell lines are BG01, BG02, BG03, BG04, CY12, CY30, CY92, CY10, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WA01, UCSF4, NYUES1, NYUES2, NYUES3, NYUES4, NYUES5, NYUES6, NYUES7, UCLA 1, UCLA 2, UCLA 3, WA077 (H7), WA09 (H9), WA13 (H13), WA14 (H14), HUES 62, HUES 63, HUES 64, CT1, CT2, CT3, CT4, MA135, Eneavour-2, WIBR1, WIBR2, WIBR3, WIBR4, WIBR5, WIBR6, HUES 45, Shef 3, Shef 6, BJNhem19, BJNhem20, SA001, SA001.

Induced pluripotent stem cells (iPSCs; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as omentum) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kf14 and c-Myc/1-Myc in omental cells.

Examples of specialized retinal cells which may be used to fabricate the construct include but are not limited to retinal pigment epithelial (RPE) cells and photoreceptors. Other cells which may be used to fabricate the retinal construct include, but are not limited to endothelial cells, fibroblasts, neurons and ganglion cells.

Methods of differentiating RPE cells from pluripotent stem cells are known in the art-see for example Nair et al Appl Sci (Basel). 2021 March; 11 (5): 2154, the contents of which are incorporated herein by reference.

The term “photoreceptors” as used herein refers to biological cells that are capable of phototransduction. The photoreceptors of this aspect of the present invention may be rods and/or cones. In one embodiment, the photoreceptors express nestin. Preferably, upon implantation within an eye, they exhibit functional activities similar to those of native photoreceptors.

Methods of differentiating photoreceptor cells from pluripotent stem cells are known in the art—see for example Nair et al Appl Sci (Basel). 2021 March; 11 (5): 2154, the contents of which are incorporated herein by reference.

The photoreceptors may be cultured in agents known to further promote the differentiation and or survival of photoreceptors. Such agents include, but are not limited to FGF, shh, noggin, antagonists of Wnt (Dkk1 or IWR1e), nodal antagonists (Lefty-A), retinoic acid, taurine, GSK3b inhibitor (CHIR99021) and notch inhibitor (DAPT).

“Retinal pigment epithelium cells”, “RPE cells”, “RPEs”, which may be used interchangeably as the context allows, refers to cells of a cell type functionally similar to that of native RPE cells which form the pigment epithelium cell layer of the retina (e.g., upon implantation within an eye, they exhibit functional activities similar to those of native RPE cells).

According to one embodiment, the RPE cell expresses at least one, two, three, four or five markers of mature RPE cells. Such markers include, but are not limited to CRALBP, RPE65, PEDF, PMEL17, Bestrophin, ZO-1 and tyrosinase. Optionally, the RPE cells may also express a marker of an RPE progenitor—e.g., MITF. In another embodiment, the RPE cells express PAX-6. In another embodiment, the RPE cells express at least one marker of a retinal progenitor cell including, but not limited to Rx, OTX2 or SIX3. Optionally, the RPE cells express either SIX6 and/or LHX2. Optionally, the RPE cells express CD31 and PAX6.

In one embodiment, the RPE cells are connected via tight junctions (as determined by expression of the tight junction marker ZO1).

In another embodiment, the RPE cells form a monolayer and optionally display a basolateral localization of BEST1.

As used herein the phrase “markers of mature RPE cells” refers to antigens (e.g., proteins) that are elevated (e.g., at least 2 fold, at least 5 fold, at least 10 fold) in mature RPE cells with respect to non RPE cells or immature RPE cells.

As used herein the phrase “markers of RPE progenitor cells” refers to antigens (e.g., proteins) that are elevated (e.g., at least 2 fold, at least 5 fold, at least 10 fold) in RPE progenitor cells with respect to non RPE cells.

According to another embodiment, the RPE cells have a morphology similar to that of native RPE cells which form the pigment epithelium cell layer of the retina i.e. pigmented and having a characteristic polygonal shape.

According to a particular embodiment, the construct is vascularized i.e. comprises at least one tubular structure generated from endothelial cells, and optionally fibroblasts.

The endothelial cells may be human embryonic stem cell (hESC)-derived endothelial cells (Levenberg, et al., Proc Natl Acad Sci USA (2002) 99, 4391-4396, the contents of which are incorporated by reference herein), or primary endothelial cells cultured from e.g. human umbilical vein (HUVEC), or biopsy-derived endothelial cells such as from the aorta or umbilical artery. The endothelial cells of the constructs of the present invention may also be derived from humans (either autologous or non-autologous) e.g. from the blood or bone marrow. In addition the endothelial cells may be derived from other mammals, for example, humans, mice or cows. For example, endothelial cells may be retrieved from bovine aortic tissue.

In one embodiment, human embryonic endothelial cells are produced by culturing human embryonic stem cells in the absence of LIF and bFGF to stimulate formation of embryonic bodies, and isolating PECAM1 positive cells from the population. HUVEC may be isolated from tissue according to methods known to those skilled in the art or purchased from cell culture laboratories such as Cambrex Biosciences or Cell Essentials.

Promotion of 3D endothelial structures may also be enhanced by addition of fibroblast cells (e.g. human embryonic fibroblasts). Fibroblasts may be isolated from tissue according to methods known to those skilled in the art (e.g. obtained from E-13 ICR embryos) or purchased from cell culture laboratories such as Cambrex Biosciences or Cell Essentials.

According to some of any of the embodiments described herein, the matrix of which at least a portion of the cellularized engineered construct is formed is an ECM-based hydrogel, as described herein in any of the respective embodiments and any combination thereof.

Herein and in the art, the term “hydrogel” describes a three-dimensional fibrous network containing at least 20%, typically at least 50%, or at least 80%, and up to about 99.99% (by mass) water or an aqueous solution. A hydrogel can be regarded as a material which is mostly water, yet behaves like a solid or semi-solid due to a three-dimensional interconnected solid-like fibrous network, within the liquid dispersing medium.

As used herein the phrase “fibrous network” refers to a set of connections formed between a plurality of fibrous components. Herein, the fibrous components are optionally composed of a plurality of polymeric chains, typically fibrillar polymeric chains, which can be made of polymeric biological materials (e.g., macromolecules) such as peptides, proteins, oligonucleotides and nucleic acids and/or of synthetic materials, preferably biocompatible polymers.

Hydrogels may take a physical form that ranges from soft, brittle and weak to hard, elastic and tough material. Soft hydrogels may be characterized by rheological parameters including elastic and viscoelastic parameters, while hard hydrogels are suitably characterized by tensile strength parameters, elastic, storage and loss moduli, as these terms are known in the art.

The softness/hardness of a hydrogel is governed inter alia by the chemical composition of the polymeric chains, the type of the interaction between the polymeric chains (type of cross-linking), the degree of cross-linking (number of interconnected links between the chains), the aqueous media content and composition, and temperature.

The interconnecting links between the chains in the fibrous network can be chemical or physical, and are also referred to as chemical cross-linking and physical cross-linking, respectively.

By chemical cross-linking, it is meant the at least a portion of the chains composing the network are covalently linked to one another.

By physical cross-linking, it is meant that the chains composing the network are linked to one another either physically, for example, by means of entanglement, and/or chemically, via non-covalent interactions (e.g., electrostatic and/or hydrogen and/or aromatic interactions).

The degree of cross-linking describes the mol % of chains that are interconnected to one another chemically, via covalent bonds.

According to some embodiments, a hydrogel as described herein, prior to contacting the reinforcing agent, features a degree of crosslinking, as defined herein, of no more than 10%, or of no more than 5%, or of no more than 2%, or of no more than 1%, or null.

The terrm “ECM hydrogel” refers to a hydrogel which comprises components of the extracellular matrix (ECM) including, but not limited to collagen, hyaluronic acid and elastin, that is, the fibrous network is composed mainly of fibrillar chains of ECM proteins. Typically, the hydrogel is derived from tissue comprising ECM. According to embodiments of the invention, the ECM hydrogel is viscoelastic, thermoresponsive, has low swelling ratio and is biocompatible and degradable.

In one embodiment, the ECM hydrogel is not Matrigel.

In another embodiment, the ECM hydrogel is derived from decellularized mammalian tissue. Exemplary components of an ECM-based hydrogel derived from decellularized mammalian tissue include: collagen type I, II, III, IV, V, VI, laminin, elastin, fibronectin and glycosaminoglycans (sulfated and nonsulfated).

In additional embodiments, the ECM hydrogel is derived from decellularized mammalian tissue and is not enzymatically solubilized and neutralized to physiologic pH and temperature. Examples of such ECM hydrogels include decellularized human lipoaspirate, intervertebral disc and devitalized cartilage.

In additional embodiments, the ECM hydrogel is derived from decellularized mammalian tissue and is enzymatically solubilized and neutralized to physiologic pH and temperature.

Exemplary tissues which may be decellularized to form ECM hydrogels include, but are not limited to small intestinal submucosa (SIS), urinary bladder matrix (UBM), adipose tissue, bone, cartilage, heart, kidney, liver, lung, skeletal muscle, tendon, umbilical cord. According to a particular embodiment, the tissue is omentum.

In one embodiment, the tissue is porcine or bovine tissue. In another embodiment, the tissue is human tissue (e.g. human omental tissue).

Methods of decellularizing tissues are known in the art and are further described in Saldin et al., Acta Bimater, 2017 February 49,pages 1-15, Machluf et al (WO 2014/037942), Yang (EP 222191) and Badylak et al (US 2008/0260831) and Dvir et al., US 2020/0101198-A1) the contents of which are incorporated herein by reference.

Following decellularization, the decellularized tissue may be dehydrated e.g. lyophilized. The lyophilized, decellularized tissue may be cut into small pieces, e.g. crumbled, or milled into a powder and then subjected to proteolytic digestion. The digestion is effected under conditions that allow the proteolytic enzyme to digest and solubilize the ECM (e.g. by cleaving the telopeptide bonds of the collagen triple helix structure to unravel collagen fibril aggregates). Thus, according to one embodiment, the digestion is carried out in the presence of an acid (e.g. hydrochloric acid or acetic acid) so as to obtain a pH of about 1-4.

Proteolytic digestion according to this aspect of the present invention can be effected using a variety of proteolytic enzymes. Non-limiting examples of suitable proteolytic enzymes include trypsin, pepsin, collaganease and pancreatin which are available from various sources such as from Sigma (St Louis, MO, USA) and combinations thereof. Matrix degrading enzymes such as matrix metalloproteinases are also contemplated.

It should be noted that the concentration of the digestion solution and the incubation time therein depend on the type of tissue being treated and the size of tissue segments utilized and those of skilled in the art are capable of adjusting the conditions according to the desired size and type of tissue. Preferably, the tissue segments are incubated for at least about 20 hours, more preferably, at least about 24 hours. Preferably, the digestion solution is replaced at least once such that the overall incubation time in the digestion solution is at least 40-48 hours.

Once the decellularized ECM is solubilized (when the liquid is homogeneous with no visible particles), the pH of the solution is increased so as to irreversibly inactivate the proteolytic enzyme (e.g. to about pH 7). The decellularized, solubilized tissue (e.g. omentum) may be stored at this stage at temperatures lower than 20° C.—for example 4° C. so that the decellularized ECM remains in solution.

Typically the ECM hydrogel has a DNA content per dry weight of hydrogel being less than 50 ng per dry weight of hydrogel, less than 40 ng per dry weight of hydrogel, or even less than 30 ng per dry weight of hydrogel.

According to still another embodiment, the diameter of the fibers in the ECM based hydrogel is between 5-500 nm (for example between 20-400 nm).

For a 1% gel, the storage modulus G′(t=0;Pa) may be between 10-20 for example 16-18. For a 1% gel, the storage modulus G′(t=0.5;Pa) may be between 100-200 for example 120-160. For a 1% gel, the storage modulus G′(t=0.95;Pa) may be between 100-200 for example 120-160. For a 1.5% gel, the storage modulus G′(t=0;Pa) may be between 10-50 for example 30-40. For a 1.5% gel, the storage modulus G′(t=0.5;Pa) may be between 200-500 for example 300-400. For a 1.5% gel, the storage modulus G′(t=0.95;Pa) may be between 200-500 for example 250-450.

For a 1% gel, the loss modulus G″(t=0;Pa) may be between 5-20 for example 10-15. For a 1% gel, the loss modulus G″(t=0.5;Pa) may be between 10-100 for example 20-50. For a 1% gel, the loss modulus G″(t=0.95;Pa) may be between 10-100 for example 20-50. For a 1.5% gel, the loss modulus G″(t=0;Pa) may be between 10-50 for example 20-40. For a 1.5% gel, the loss modulus G″(t=0.5;Pa) may be between 10-100 for example 40-70. For a 1.5% gel, the loss modulus G″(t=0.95;Pa) may be between 10-100 for example 50-80.

The swelling ratio of the hydrogels of this aspect of the present invention are typically between 30-50, with the exact values depending on the length of time the hydrogel has been swollen and the percent precursor present in the hydrogel. Typically, the higher the precursor concentration in the hydrogel, the lower the swelling ratio.

In some embodiments, the ECM hydrogel is a thermoreversible gel (also known as thermo-responsive gel or thermogel). In some embodiments, a suitable thermoreversible hydrogel is a liquid at room temperature. In some embodiments, a suitable thermoreversible hydrogel is a solid at physiological temperature. In specific embodiments, the gelation temperature (Tgel) of a suitable hydrogel is about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., including increments therein. In certain embodiments, the Tgel of a suitable hydrogel is about 10° C. to about 40° C. In further embodiments, the Tgel of a suitable hydrogel is about 20° C. to about 30° C.

The engineered constructs may be fabricated using any method known in the art.

In one embodiment, the cells are combined with the ECM hydrogels and seeded on a solid or semi-solid scaffold. The architecture and 3D shape of the scaffold ultimately dictates the size and shape of the construct.

As used herein, the term “scaffold” refers to synthetic scaffolds such as polymer scaffolds, and non-synthetic scaffolds such as pre-formed extracellular matrix layers, dead cell layers, and decellularized tissues, and any other type of pre-formed scaffold that is integral to the physical structure of the engineered tissue and/or organ and not able to be removed from the tissue and/or organ without damage/destruction of the tissue and/or organ.

In some embodiments, at least one layer of the constructs are bioprinted. According to a specific embodiment, the first layer of the constructs comprising endothelial cells is bioprinted. According to a specific embodiment, tubular structures comprising endothelial cells are bioprinted. The endothelial cells may be printed using a bioink which comprises the ECM hydrogel. Support media may be used which provide stability to the structure when printed. Once the structure is stabilized and more rigid, the support medium may be removed (e.g. dissolved). Optionally, the support medium comprises calcium alginate hydrogel particles. Other support media are further described herein below. The alginate may be removed using an enzymatic process (e.g. using alginate lyase), once the structure is stabilized.

In other embodiments, the engineered constructs are entirely bioprinted, that is, are generated entirely by bioprinting, in particular 3D bioprinting, as described herein in any of the respective embodiments and any combination thereof.

### Bioprinting Techniques:

A bioprinting method and a corresponding system can be any of the methods and systems known in the art for performing additive manufacturing. A suitable method and system can be selected upon considering its printing capabilities, which include resolution, deposition speed, scalability, bio-ink compatibility and ease-of-use.

Exemplary suitable bioprinting systems usually contain a temperature-controlled material handling with a dispensing system and stage (a receiving medium), and a movement along the x, y and z axes directed by a CAD-CAM software. A curing source (e.g., a light or heat source) which applies a curing energy (e.g., by applying light or heat radiation) or a curing condition to the deposition area (the receiving medium) so as to promote hardening or solidification of the formed layers and/or a humidifier, can also be included in the system. There are printers that use multiple dispensing heads to facilitate a serial dispensing of several materials.

3D bioprinting is an additive manufacturing methodology which uses biological materials, optionally in combination with chemicals and/or cells, that are printed layer-by-layer with a precise positioning and a tight control of functional components placement to create a 3D structure.

Inherent to 3D printing in general is that the mechanical properties of the printing media (the dispensed material; bioink) are different from the post-printed cured (hardened; solidified) material.

Different technologies have been developed for 3D bioprinting, including 3D Inkjet printing, Extrusion printing, Laser-assisted printing, digital light processing, and Projection stereolithography [see, for example, Murphy S V, Atala A, Nature Biotechnology. 2014 32 (8).; Miller J S, Burdick J. ACS Biomater. Sci. Eng. 2016, 2, 1658-1661]. Each technology has its different requirements for the dispensed building material (also referred to herein as printing media), which is derived from the specific application mechanism and the curing/gelation process required to maintain the 3D structure of the scaffold post printing.

The following describes embodiments of additive manufacturing processes and methodologies for which the method as described herein can be employed.

According to an aspect of some embodiments of the present invention, there is provided a process (a method) of additive manufacturing (AM) of a three-dimensional object. According to embodiments of this aspect, the method is effected by sequentially forming a plurality of layers in a configured pattern corresponding to the shape of the object, thereby forming the object. According to some embodiments of this aspect, formation of each layer is effected by dispensing at least one uncured building material, and exposing the dispensed building material to a curing condition to thereby form a hardened (cured; solidified) material. According to some embodiments of this aspect, formation of each layer is effected by exposing a layer of uncured building material to a curing condition, and the method is effected by sequentially exposing, in a layer-wise manner, an uncured building material to a curing condition.

Herein throughout, the phrase “building material” encompasses the phrases “uncured building material” or “uncured building material formulation” and collectively describes the materials that are dispensed by sequentially forming the layers, as described herein. This phrase encompasses uncured materials which form the final object, namely, one or more uncured modeling material formulation(s), or bioink composition(s) and optionally also uncured materials used to form a support, namely uncured support material formulations. The building material can also include non-curable materials that preferably do not undergo (or are not intended to undergo) any change during the process, for example, biological materials or components and/or other agents or additives as described herein.

The building material that is used to sequentially form the layers as described herein is also referred to herein interchangeably as “printing medium” or “bioprinting medium” “bioink composition” or “bioink”.

An uncured building material can comprise one or more modeling material formulations, and can be utilized such that different parts of the object are made upon hardening of different modeling formulations, and hence are made of different hardened modeling materials or different mixtures of hardened modeling materials.

The method of the present embodiments manufactures three-dimensional objects in a layerwise manner by forming a plurality of layers in a configured pattern corresponding to the shape of the object.

Each layer is formed by an additive manufacturing apparatus which scans a two-dimensional surface and patterns it. While scanning, the apparatus visits a plurality of target locations on the two-dimensional layer or surface, and decides, according to a pre-set algorithm, for each target location or a group of target locations, whether or not the target location or group of target locations is to be occupied by a building material, and which type of a building material is to be delivered thereto. The decision is made according to a computer image of the surface.

When the AM is by three-dimensional inkjet printing, an uncured building material, as defined herein, is dispensed from a dispensing head having a set of nozzles to deposit building material in layers on a supporting structure. The AM apparatus thus dispenses building material in target locations which are to be occupied and leaves other target locations void. The apparatus typically includes a plurality of dispensing heads, each of which can be configured to dispense a different building material (for example, different modeling material formulations, each containing a different biological component; or each containing a different curable material; or each containing a different concentration of a curable material, and/or different support material formulations). Thus, different target locations can be occupied by different building materials (e.g., a modeling formulation and/or a support formulation, as defined herein).

The final three-dimensional object is made of the hardened modeling material or a combination of hardened modeling materials or a combination of hardened modeling material/s and support material/s or modification thereof. All these operations are well-known to those skilled in the art of additive manufacturing (also known as solid freeform fabrication).

In some exemplary embodiments of the invention an object is manufactured by dispensing a building material that comprises two or more different modeling material formulations, each modeling material formulation from a different dispensing head of the AM apparatus. The modeling material formulations are optionally and preferably deposited in layers during the same pass of the dispensing heads. The modeling material formulations and/or combination of formulations within the layer are selected according to the desired properties of the object.

An exemplary process according to some embodiments of the present invention starts by receiving 3D printing data corresponding to the shape of the object. The data can be received, for example, from a host computer which transmits digital data pertaining to fabrication instructions based on computer object data, e.g., in a form of a Standard Tessellation Language (STL) or a StereoLithography Contour (SLC) format, Virtual Reality Modeling Language (VRML), Additive Manufacturing File (AMF) format, Drawing Exchange Format (DXF), Polygon File Format (PLY), Digital Imaging and Communications in Medicine (DICOM) or any other format suitable for Computer-Aided Design (CAD). In some embodiments, the object is bio-printed in a size and shape that corresponds to a subject's anatomy. These parameters can be obtained for example from imaging data such that the method can further comprise acquiring an imaging data for a size and shape of the object, and bioprinting the object in accordance with the imaging data. The imaging data can be of a subject to be treated with the object or of an exemplary data, so as to serve as a computed model for the additive manufacturing.

The process continues by dispensing the building material as described herein in layers, on a receiving medium, using one or more dispensing (e.g., printing) heads, according to the printing data.

The dispensing can be in a form of droplets, or a continuous stream, depending on the additive manufacturing methodology employed and the configuration of choice.

The receiving medium can be a tray of a printing system, or a supporting article or medium made of, or coated by, a biocompatible material, such as support media or articles commonly used in bioprinting, or a previously deposited layer.

In some embodiments, the receiving medium comprises a sacrificial hydrogel or other biocompatible material as a mold to embed the printed object, and is thereafter removed by chemical, mechanical or physical (e.g., heating or cooling) means. Such sacrificial hydrogels can be made of, for example, a Pluronic material or of Gelatin. An exemplary sacrificial hydrogel is made of calcium alginate, xanthan gum and gluconic acid δ-lactone

Once the uncured building material is dispensed on the receiving medium according to the 3D data, the method optionally and preferably continues by hardening the dispensed formulation(s). In some embodiments, the process continues by exposing the deposited layers to a curing condition. Preferably, the curing condition is applied to each individual layer following the deposition of the layer and prior to the deposition of the previous layer.

As used herein throughout, the term “curing” describes a process in which a formulation is hardened. The hardening of a formulation typically involves an increase in a viscosity of the formulation and/or an increase in a storage modulus of the formulation (G′). In some embodiments, a formulation which is dispensed as a liquid becomes solid or semi-solid (e.g., gel) when hardened. A formulation which is dispensed as a semi-solid (e.g., soft gel) becomes solid or a harder or stronger semi-solid (e.g., strong gel) when hardened.

The term “curing” as used herein encompasses, for example, polymerization of monomeric and/or oligomeric materials and/or (e.g., physical) cross-linking of polymeric chains (for example, ECM protein chains). The product of a curing reaction can therefore be a (e.g., physically) cross-linked material. This term, as used herein, encompasses also partial curing, for example, curing of at least 20% or at least 30% or at least 40% or at least 50% or at least 60% or at least 70% of the formulation, in addition to curing of 100% of the formulation.

Herein, the phrase “a condition that affects curing” or “a condition for inducing curing”, which is also referred to herein interchangeably as “curing condition” or “curing inducing condition” describes a condition which, when applied to a formulation that contains a curable material, induces a curing as defined herein. Such a condition can include, for example, application of a curing energy, as described hereinafter to the curable material(s), and/or simple solidification at ambient or physiological environment.

In some embodiments, the condition includes a temperature change. Thus, in some embodiments, the bioink when used for printing is in a liquid form, e.g. printed at a temperature below 37° C., e.g., lower than 30, or lower than 25, or lower than 20, or at an ambient temperature (e.g., room temperature of about 25° C.), or at lower temperatures, typically upon cooling the system to a temperature lower than 20, or lower than 15, or lower than 10, or lower than 5, typically 4 or 0,° C. The bioink hardens (or gelifies) at a temperature of 37° C., such that the curing condition comprises exposing the deposited hydrogel to a temperature of about 37° C., by, e.g., heating.

Some embodiments contemplate the fabrication of an object by depositing or dispensing different formulations from different dispensing heads. These embodiments provide, inter alia, the ability to select formulations from a given number of formulations and define desired combinations of the selected formulations and their properties.

According to the present embodiments, the spatial locations of the deposition of each formulation with the layer are defined, either to effect occupation of different three-dimensional spatial locations by different formulations, or to effect occupation of substantially the same three-dimensional location or adjacent three-dimensional locations by two or more different formulations so as to allow post deposition spatial combination of the formulations within the layer.

The present embodiments thus enable the deposition of a broad range of material combinations, and the fabrication of an object which may consist of multiple different combinations of modeling material formulations, in different parts of the object, according to the properties desired to characterize each part of the object.

A system utilized in additive manufacturing may include a receiving medium and one or more dispensing heads. The receiving medium can be, for example, a fabrication tray that may include a horizontal surface to carry the material dispensed from the printing head. In some embodiments, the receiving medium is made of, or coated by, a biocompatible material, as described herein.

The dispensing head may be, for example, a printing head having a plurality of dispensing nozzles arranged in an array of one or more rows along the longitudinal axis of the dispensing head. The dispensing head may be located such that its longitudinal axis is substantially parallel to the indexing direction.

The additive manufacturing system may further include a controller, such as a microprocessor to control the AM process, for example, the movement of the dispensing head according to a pre-defined scanning plan (e.g., a CAD configuration converted to a Standard Tessellation Language (STL) format and programmed into the controller). The dispensing head may include a plurality of jetting nozzles. The jetting nozzles dispense material onto the receiving medium to create the layers representing cross sections of a 3D object.

In addition to the dispensing head, there may be a source of curing energy, for curing the dispensed building material. The curing energy is typically radiation, for example, UV radiation or heat radiation. Alternatively, there may be means for providing a curing condition other than electromagnetic or heat radiation, for example, means for heating or cooling the dispensed building material or for contacting it with a reagent that promotes curing.

According to the present embodiments, the additive manufacturing method described herein is for bioprinting a biological object.

As used herein, “bioprinting” means practicing an additive manufacturing process while utilizing one or more bio-ink formulation(s) that comprise(s) biological components, as described herein, via a methodology that is compatible with an automated or semi-automated, computer-aided, additive manufacturing system as described herein (e.g., a bioprinter or a bioprinting system).

Herein throughout, the phrase “modeling material formulation”, which is also referred to herein interchangeably as “modeling formulation” or “modeling material composition” or “modeling composition”, or simply as a “formulation”, or a “composition”, describes a part or all of the uncured building material (printing medium) which is dispensed so as to form the final object, as described herein. The modeling formulation is an uncured modeling formulation, which, upon exposure to a curing condition, forms the object or a part thereof.

In the context of bioprinting, an uncured building material comprises at least one modeling formulation that comprises one or more biocompatible or biological components or materials (e.g., an ECM-based hydrogel as described herein and/or cells as described herein), and is also referred to herein and in the art as “bioink” or “bioink formulation” or “bioink composition”.

In some embodiments, the bioprinting comprises sequential formation of a plurality of layers of the uncured building material in a configured pattern, preferably according to a three-dimensional printing data, as described herein. At least one, and preferably most or all, of the formed layers (before hardening or curing) comprise(s) one or more biological component(s) as described herein (e.g., an ECM-based hydrogel and/or cells as described herein). Optionally, at least one of the formed layers (before hardening or curing) comprises one or more non-biological curable materials, and/or non-curable biological or non-biological components, preferably biocompatible materials which do not interfere (e.g., adversely affect) with the biological and/or structural features of the biological components (e.g., collagen) in the printing medium and/or bio-ink.

In some embodiments, the components in the bioink or the printing medium, e.g., non-curable and curable materials, and/or the curing condition applied to effect curing, are selected such that they do not significantly affect structural and/or functional properties of the biological components in the bio-ink or printing medium.

In some of any of the embodiments described herein, the building material (e.g., the printing medium) comprises modeling material formulation(s) (e.g., a bioink composition as described herein) and optionally support material formulation(s), and all are selected to include materials or combination of materials that do not interfere with the biological and/or structural features of the biological components.

In some of any of the embodiments described herein, the bioprinting method is configured to effect formation of the layers under conditions that do not significantly affect structural and/or functional properties of the biological components in the bioink composition.

In some embodiments, a bioprinting system for effecting a bioprinting process/method as described herein is configured so as to allow formation of the layers under conditions that do not significantly affect structural and/or functional properties of the biological components in the bio-ink.

In some of any of the embodiments described herein, the additive manufacturing (e.g., bioprinting) process and system are configured such that the process parameters (e.g., temperature, shear forces, shear strain rate) do not interfere with (do not substantially affect) the functional and/or structural features of the biological components.

In some of any of the embodiments described herein, the additive manufacturing process (bioprinting) is performed while applying a shear force that does not adversely affect structural and/or functional properties of biological components (e.g., cells). Applying the shear force can be effected by passing the building material (e.g., at least a modeling material formulation that comprises a biological component as described herein, a bioink composition) through the dispensing head, and is to be regarded also as subjecting the building material to shear force.

Some embodiments of the present invention allow to perform AM bioprinting processes under conditions that do not affect the functional and/or structural features of biological components included in the bio-ink (e.g., at low shear force and room temperature or a physiological temperature), while maintaining the required fluidity (a viscosity that imparts fluidity, e.g., lower than 10,000 centipoises or lower than 5,000 centipoises, or lower than 2,000 centipoises), and while further maintaining the curability of the dispensed building material.

The following describes exemplary AM bioprinting methodologies that are usable in the context of embodiments of the present invention.

A bioprinting method and a corresponding system can be any of the methods and systems known in the art for performing additive manufacturing, and exemplary such systems and methods are described hereinabove. A suitable method and system can be selected upon considering its printing capabilities, which include resolution, deposition speed, scalability, bio-ink compatibility and ease-of-use.

Exemplary suitable bioprinting systems usually contain a dispensing system (either equipped with temperature control module or at ambient temperature), and stage (a receiving medium), and a movement along the x, y and z axes directed by a CAD-CAM software. A curing source (e.g., a light or heat source) which applies a curing energy (e.g., by applying light or heat radiation) or a curing condition to the deposition area (the receiving medium) so as to promote curing of the formed layers and/or a humidifier, can also be included in the system. There are printers that use multiple dispensing heads to facilitate a serial dispensing of several materials.

Generally, bioprinting can be effected using any of the known techniques for additive manufacturing. The following lists some exemplary additive manufacturing techniques, although any other technique is contemplated.

### 3D Inkjet Printing:

3D Inkjet printing is a common type of 3D printer for both non-biological and biological (bioprinting) applications. Inkjet printers use thermal or acoustic forces to eject drops of liquid onto a substrate, which can support or form part of the final construct. In this technique, controlled volumes of liquid are delivered to predefined locations, and a high-resolution printing with precise control of (1) ink drops position, and (2) ink volume, which is beneficial in cases of microstructure-printing or when small amounts of bioreactive agents or drugs are added, is received. Inkjet printers can be used with several types of ink, for example, comprising multiple types of biological components and/or bioactive agents. Furthermore, the printing is fast and can be applied onto culture plates.

A bioprinting method that utilizes a 3D inkjet printing system can be operated using one or more bio-ink modeling material formulations as described herein, and dispensing droplets of the formulation(s) in layers, on the receiving medium, using one or more inkjet printing head(s), according to the 3D printing data.

### Extrusion Printing:

This technique uses continuous beads of material rather than liquid droplets. These beads of material are deposited in 2D, the stage (receiving medium) or extrusion head moves along the z axis, and the deposited layer serves as the basis for the next layer. The most common methods for biological materials extrusion for 3D bioprinting applications are pneumatic or mechanical dispensing systems

### Stereolithography (SLA) and Digital Light Processing (DLP):

SLA and DLP are additive manufacturing technologies in which an uncured building material in a bath or vat is converted into hardened material(s), layer by layer, by selective curing using a light source while the uncured material is later separated/washed from the hardened material. SLA is widely used to create models, prototypes, patterns, and production parts for a range of industries including for Bioprinting. DLP differs from laser-based SLA in that DLP uses a projection of ultraviolet (UV) light (or visible light) from a digital projector to flash a single image of the layer across the entire uncured material at once. One of the key components of DLP is a digital micromirror device (DMD) chip, which is typically composed of an array of reflective aluminum micromirrors that redirect incoming light from the UV source to project an image of a designed pattern. For achieving a high-resolution structure, parameters such as the curing time of each layer, layer thickness, and intensity of the UV light should be tuned, for example, by controlling the concentration and types of the curable materials, the photoabsorber and/or the photoinitiator.

### Laser-Assisted Printing:

Laser-assisted printing technique, in the version adopted for 3D bioprinting, is based on the principle of laser-induced forward transfer (LIFT), which was developed to transfer metals and is now successfully applied to biological material. The device consists of a laser beam, a focusing system, an energy absorbing/converting layer and a biological material layer (e.g., cells and/or hydrogel) and a receiving substrate. A laser assisted printer operates by shooting a laser beam onto the absorbing layer which convert the energy into a mechanical force which drives tiny drops from the biological layer onto the substrate. A light source is then utilized to cure the material on the substrate.

Laser assisted printing is compatible with a series of viscosities and can print mammalian cells without affecting cell viability or cell function. Cells can be deposited at a density of up to 10⁸ cells/ml with microscale resolution of a single cell per drop.

### Electrospinning:

Electrospinning is a fiber production technique, which uses electric force to draw charged threads of polymer solutions, or polymer melts.

According to some of any of the embodiments described herein, the additive manufacturing (bioprinting) is an extrusion-based bioprinting, as described herein.

Herein throughout, in the context of bioprinting, the term “object” describes a final product of the additive manufacturing which comprises, in at least a portion thereof, a biological component. This term refers to the product obtained by a bioprinting method as described herein, after removal of the support material, if such has been used as part of the uncured building material.

In some embodiments, the engineered constructs are comprised essentially of cellular material and ECM hydrogels (i.e. bioink) prior to reinforcing. In various further embodiments, the cell-comprising portions of the engineered constructs consist of 30, 35, 40, 45, 50, 55, 60, 65, 70, 75, 80, 85, 90, 95, 96, 97, 98, 99, 99.5, 99.9, and 100% cellular material, including increments therein, prior to reinforcing. In one embodiment, at least 20%, 30%, 40%, 50%, 60%, 70%, 80%, 90% of the cells of the construct are mature cells prior to contacting with the reinforcing agent. In other various embodiments, the cell-comprising portions of the engineered constructs consist of 30, 35, 40, 45, 50, 55, 60, 65, 70, 75, 80, 85, 90, 95, 96, 97, 98, 99, 99.5, 99.9, and 100% cellular material, including increments therein, at the time of implantation. In some embodiments, the engineered retinal constructs are cohered and/or adhered aggregates of cells prior to reinforcing (i.e. the retinal constructs are cultured in a culture medium for a length of time such that at least a portion of the cells interact biologically with each other and are cohered and/or adhered prior to reinforcing), In some embodiments, a portion of the non-cellular components are removed prior to reinforcing. In further embodiments, the non-cellular components are removed by physical, chemical, or enzymatic means. In some embodiments, a proportion of the non-cellular components remains associated with the cellular components at the time of reinforcing. In some embodiments, the non-cellular components are selected from a group that includes: hydrogels, surfactant polyols, thermo-responsive polymers, hyaluronates, alginates, collagens, or other biocompatible natural or synthetic polymers.

In one embodiment, the bioink comprises a single-cell suspension in the ECM-based hydrogel. In another embodiment, the bioink comprises clusters of cells in the ECM based hydrogel.

In some embodiments, the bio-ink further comprises an agent that encourages cell adhesion.

In some embodiments, the bio-ink further comprises an agent that inhibits cell death (e.g., necrosis, apoptosis, or autophagocytosis). In some embodiments, the bio-ink further comprises an anti-apoptotic agent. Agents that inhibit cell death include, but are not limited to, small molecules, antibodies, peptides, peptibodies, or combination thereof. In some embodiments, the agent that inhibits cell death is selected from: anti-TNF agents, agents that inhibit the activity of an interleukin, agents that inhibit the activity of an interferon, agents that inhibit the activity of an GCSF (granulocyte colony-stimulating factor), agents that inhibit the activity of a macrophage inflammatory protein, agents that inhibit the activity of TGF-B (transforming growth factor B), agents that inhibit the activity of an MMP (matrix metalloproteinase), agents that inhibit the activity of a caspase, agents that inhibit the activity of the MAPK/INK signaling cascade, agents that inhibit the activity of a Src kinase, agents that inhibit the activity of a JAK (Janus kinase), or a combination thereof. In some embodiments, the bio-ink comprises an anti-oxidant.

In some embodiments, the bio-ink further comprises an extrusion compound (i.e, a compound that modifies the extrusion properties of the bio-ink). Examples of extrusion compounds include, but are not limited to gels, hydrogels, peptide hydrogels, amino acid-based gels, surfactant polyols (e.g., Pluronic F-127 or PF-127), thermo-responsive polymers, hyaluronates, alginates, extracellular matrix components (and derivatives thereof), collagens, other biocompatible natural or synthetic polymers, nanofibers, and self-assembling nanofibers.

In some embodiments of any of the embodiments described herein, the bio-ink composition is devoid of a curing agent that promotes chemical cross-linking of the hydrogel components and/or of curable materials that can undergo chemical cross-linking upon exposure to a curing condition (e.g., a temperature change, for example, heating). By “devoid of” it is meant less than 5%, or less than 2% preferably less than 1%, or less than 0.5%, or less than 0.1%, or less than 0.05%, or less than 0.01%, or null.

### Reinforcing Agent:

According to embodiments of the present invention, a reinforcing agent is a biocompatible small molecule that is capable of penetrating a cellularized engineered tissue and thereby distribute substantially homogeneously throughout the tissue, both internally and externally. According to some embodiments, the biocompatible small-molecule reinforcing agent is capable of chemically interacting with one or more materials that form the construct matrix of the engineered tissue. According to some embodiments, the biocompatible small-molecule reinforcing agent is capable of chemically interacting with the materials that form the matrix under conditions that maintain viability of the cells, to thereby provide a chemically cross-linked matrix. According to some embodiments, the reinforcing agent acts as a cross-linking agent, which chemically cross-link polymeric chains in the matrix (e.g., protein chains in an ECM-based hydrogel as described herein).

By “biocompatible” it is meant that it poses limited risk of injury or toxicity to organisms that it contacts.

By “small molecule” it is meant that the molecule is spatially arranged such that it can penetrate through the pores of the matrix used to construct the tissue, that is, its volume is lower by at least 5%, or by at least 10%, and preferably much lower, than the average pore size of the matrix. According to some embodiments, the reinforcing agent has a molecular weight of no more than 1,000 grams/mol, or no more than 80 grams/mol, or no more than 600 grams/mol, or no more than 500 grams/mol, or no more than 400 grams/mol, or no more than 300 grams/mol, or no more than 200 grams/mol, and can range, for example, from 50 to 1,000, or from 100 to 1,000, or from 50 to 800, or from 100 to 800, or from 50 to 600 or from 100 to 600, or from 50 to 500, or from 100 to 500, or from 50 to 400, or from 100 to 400, or from 200 to 1,000 or from 200 to 800, or from 200 to 700, or from 200 to 600, or from 200 to 500, or from 200 to 400, or from 200 to 300, grams/mol, including any intermediate values and subranges therebetween.

According to some of any of the embodiments described herein, the reinforcing agent is capable of chemically interacting with one or more of the materials that compose the ECM hydrogel (e.g., one or more ECM proteins) used to construct the tissue under conditions that do not affect the viability of the cells within the matrix and the integrity of the matrix (e.g., mechanical and morphological properties of the cellularized matrix).

By “chemically interacting” it is meant that the reinforcing agent is capable of forming one or more chemical bonds with one or more functional groups of one or more of the materials that compose the matrix, whereby the chemical bonds can be hydrogen bonds, electrostatic bonds, Van-der-Waals bonds and/or covalent bonds. Preferably, the reinforcing agent is capable of forming covalent bonds with the one or more materials that form the matrix, that is, can be covalently attached to the matrix under the indicated conditions.

The chemical interaction (covalent bond formation) is with free chemically compatible groups present in the matrix, that can interact with the reinforcing agent as described herein, under conditions that maintain viability of the cells, as described herein.

According to some of any of the embodiments described herein, the biocompatible small-molecule reinforcing agent is capable of chemically interacting, e.g., form covalent bonds, with at least 10%, at least 20%, at least 30%, at least 40% or at least 50%, of respective chemically compatible functional groups present in the ECM-based hydrogel before chemically interacting with said reinforcing agent.

Suitable conditions that do not affect the viability of the cells include a temperature around a physiological temperature, or otherwise a temperature that do not affect the viability of the cells; a lack of chemical reagents or solvents that may affect the viability of the cells; and a lack of physical means that may affect the viability of the cells and/or the integrity of a tissue formed of the cells and/or the integrity of the matrix. Preferably, the conditions include incubating the cellularized engineered tissue with an aqueous solution (e.g., a culturing medium) that comprises the reinforcing agent at a temperature around a physiological temperature (about 37° C.).

According to some embodiments of the present invention, the reinforcing agent is capable of interacting with a material that forms the matrix (e.g., an ECM protein) via a Click reaction.

Herein and in the art, a Click reaction describes a class of highly efficient, selective, and modular chemical reactions characterized by their orthogonality (compatibility with various functional groups) and high yields under mild reaction conditions.

An exemplary Click reaction according to the present embodiments is a Schiff-base reaction between an aldehyde and an amine-containing moiety such as amine, hydrazine, hydrazide and the like, as these terms are defined herein. This reaction can be carried out under mild conditions that do not affect the protein's essential characteristics (see, for example, Jansen E. F. and Olson A. C, Arch. Biochem. Biophys, 1969, 129 (1), pp. 221-7 and U.S. Pat. No. 4,904,592).

According to some the present embodiments, the reinforcing agent features at least one, and preferably two or more, groups that are capable of participating in a Click reaction, e.g., Shiff-base reaction, with free chemically compatible groups in the ECM-based matrix.

According to some the present embodiments, the reinforcing agent features at least two, or preferably more, aldehyde group(s), and is therefore capable of forming covalent bonds with free chemically-compatible groups of the one or more materials used to form the matrix, for example, free amine groups, via a Schiff-base Click reaction. According to some embodiments of the present invention, the reinforcing agent is a polyaldehyde.

As used herein, the term “polyaldehyde” describes a compound that has at least two free aldehyde groups, as this term is defined herein.

Polyaldehydes can readily interact with various groups via “Schiff-base” chemistry, to form imine bonds, under mild conditions.

Free amine groups are typically included in protein-based matrices, such as ECM-based matrices as described herein, as functional groups derived from side chains of certain amino acid residues, functional groups derived from the N-terminus or the C-terminus of a protein, and/or functional groups derived from residues that result from natural post-translational modification processes.

Free amine groups may form a part of functional moieties such as lysine residues present on the surface of ECM proteins such as collagen.

A polyaldehyde that participate in a Schiff-base reaction can act as a cross-linking agent, by being covalently attached to one or more protein chains that form the matrix.

According to some of any of the embodiments described herein, the reinforcing agent is a biocompatible small molecule compounds, as defined herein, which features one or more, preferably two or more aldehyde groups.

According to some of these embodiments, the reinforcing agent (featuring two or more aldehyde groups, e.g., a polyaldehyde) is capable of interacting with free amine groups in the ECM-based hydrogel, via Shiff-base chemistry as described herein (a Click chemistry), to thereby form covalent bonds. According to some embodiments, the reinforcing agent is capable of chemically interacting with at least 10, at least 20, at least 30, at least 40 or at least 50, %, or more, of free amine groups present in the ECM-based hydrogel (before chemically interacting with the reinforcing agent). According to some of these embodiments, the chemical interactions with the free amine groups, results in cross-linking the hydrogel by means of covalent attachment to two or more protein chains in the ECM-based hydrogel.

According to some of any of the embodiments described herein, the reinforcing agent is a modified saccharide, that features one or more, preferably two or more, aldehyde groups, that is, it is a saccharide of which one or more hydroxy groups have been oxidized and thereby converted to aldehyde(s). Such a modified saccharide is also referred to herein and in the art as an oxidized saccharide.

The term “saccharide” as used herein encompasses monosaccharides, disaccharides and oligosaccharides. The term “monosaccharide”, as used herein and is well known in the art, describes a simple form of a sugar that consists of a single saccharide molecule, which can be open-chain or cyclic (e.g., pyranose- or furanose-based), and which cannot be further decomposed by hydrolysis. Most common examples of monosaccharides include glucose (dextrose), fructose, galactose, and ribose. Monosaccharides can be classified according to the number of carbon atoms of the carbohydrate, i.e, triose, having 3 carbon atoms such as glyceraldehyde and dihydroxyacetone; tetrose, having 4 carbon atoms such as erythrose, threose and erythrulose; pentose, having 5 carbon atoms such as arabinose, lyxose, ribose, xylose, ribulose and xylulose; hexose, having 6 carbon atoms such as allose, altrose, galactose, glucose, gulose, idose, mannose, talose, fructose, psicose, sorbose and tagatose; heptose, having 7 carbon atoms such as mannoheptulose, sedoheptulose; octose, having 8 carbon atoms such as 2-keto-3-deoxy-manno-octonate; nonose, having 9 carbon atoms such as sialose; and decose, having 10 carbon atoms. Monosaccharides are the building blocks of oligosaccharides and disaccharides like sucrose (common sugar).

The term “disaccharide” describes a compound two monosaccharide units, which can be the same or different, covalently bound to one another, typically via a glucosyl bond.

The term “oligosaccharide” as used herein describes a compound that comprises three or more monosaccharide units, as these are defined herein, which can be the same or different. Preferably, the oligosaccharide comprises 3-6 monosaccharides units.

According to some of any of the embodiments described herein, the reinforcing agent is an oxidized saccharide, as defined herein, which features at least two, at least three, at least four, or more aldehyde groups; and/or which has been oxidized so as to covert at least one, preferably at least two, at least three or at least four, of its hydroxy groups, into aldehyde groups.

Methods of generating oxidized saccharides are well-known in the art. An exemplary such method is described in the Examples section that follows.

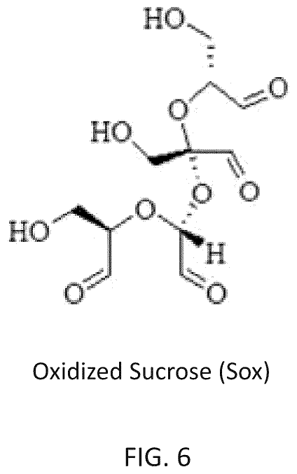

According to an exemplary embodiment, the reinforcing agent is an oxidized disaccharide, such as an oxidized sucrose (SOx), the structure of which is presented in FIG. 6.

It is to be noted that an oxidized saccharides can be selected so as to provide a desired reinforcement effect, in accordance with the materials used to form the matrix of the engineered tissue. For example, oxidized monosaccharides, or oxidized disaccharides featuring a lower molecular weight can be used to reinforce matrices that feature relatively small pores, and oxidized oligosaccharides, or oxidized monosaccharides and/or disaccharides featuring a higher molecular weight can be used to reinforce matrices that feature relatively voluminous pores.

Similarly, other compounds which feature a plurality of aldehyde groups (polyaldehydes), and which are biocompatible small molecules, can be used, and can be selected in accordance with the reinforced matrix, as above.

According to the present embodiments, the reinforcing agent is contacted with the cellularized engineered construct, subject to generation of the construct (e.g., following bioprinting of the construct, as described herein in any of the respective embodiments, or otherwise depositing a bio-ink composition as described herein, and allow it to harden, for example, by exposing to a suitable condition that promotes hardening or curing, as described herein).

According to some embodiments, the reinforcing agent is contacted with the construct under conditions as described herein.

According to some embodiments, the contacting is with a solution (e.g., aqueous solution) that comprises the reinforcing agent.

According to some embodiments, the contacting is effected by adding the reinforcing agent to a culturing medium, and subjecting the culturing medium and the construct to conditions as described herein, to thereby promote chemical interaction of the reinforcing agent with the construct. In some embodiments, the conditions comprise subjecting the culturing medium and the construct to incubation at 37° C.

According to some embodiments, the concentration of the reinforcing agent in the solution or the culturing medium is such that do not affect the viability of the cells in the construct. In some embodiments, the concentration of the reinforcing agent in the solution (e.g., culturing medium) ranges from about 0.01 to about 10%, or from about 0.01 to about 5%, or from about 0.01 to about 1%, or from about 0.01 to about 0.5%, or from about 0.01 to about 0.3%, or from about 0.01 to about 0.1%, by weight, of the total weight of the solution.

According to some embodiments of the present invention, the contacting is effected for a time period that ranges from about 1 hour to about several days (e.g., 2-14 days), or from about 12 hours to about several days, or from about 24 hours to about several days, including any intermediate values and subranges therebetween.

According to some embodiments, the concentration of the reinforcing agent and/or the contacting time is/are selected in accordance with a desired mechanical property of the final construct, that is, in accordance with a chemical cross-linking degree that provides a desired mechanical property of the final construct. Typically, for stronger or stiffer constructs, a higher concentration of the reinforcing agent and/or longer contacting times are selected, as long as the viability of cells in the construct is maintained; and for weaker or softer constructs, a lower concentration of the reinforcing agent and/or shorter contacting time are selected. A desired mechanical property of the reinforced construct and/or a corresponding degree of cross-linking can be readily determined by those skilled in the art using available data. The respective contacting time and/or concentration of the reinforcing agent can then be determined either experimentally or by means of predictive calculation tools.

According to some embodiments, the biocompatible small-molecule reinforcing agent is chemically interacted with at least 10, at least 20, at least 30, at least 40, at least 50, %, or more, % of respective chemically compatible groups (e.g., free amine groups as described herein) present in the ECM-based hydrogel (before it chemically interacts with the reinforcing agent). The number of functional groups (which are chemically compatible with the reinforcing agent) in the ECM-based matrix, which chemically interact with the reinforcing agent can be determined by selecting the concentration of the reinforcing agent and/or the contacting time as described herein.

In one embodiment, the reinforcing agent is contacted with the construct once the cells thereof adhere to one another or interact with one another (e.g. via tight junctions, gap junctions).

In another embodiment, the reinforcing agent is contacted with the construct once the construct is at least partially vascularized.

In still another embodiment, the reinforcing agent is contacted with the construct only after at least 50%, 60%, 70%, 80%, 90% of at least one of the cell types thereof express a marker associated with a mature cell type. In still another embodiment, the reinforcing agent is contacted with the construct only after at least 50%, 60%, 70%, 80% or 90% of all of the cell types thereof express a marker associated with a mature cell type.

Preferably, the reinforcing agent is contacted with the construct at least 1 day, 2 days, 3 days, 4 days, 5 days, 6 days, 1 week, 10 days, 2 weeks after seeding/printing. The amount of time may be determined by one of skill in the art according to the cell types used in the construct and the culturing conditions used. According to another aspect of the present invention there is provided an engineered cellular construct reinforced according to the methods described herein above.

According to another aspect of the invention, there is provided a cellularized engineered retinal construct comprising endothelial cells and retinal cells distributed within a chemically cross-linked ECM hydrogel, wherein the ECM hydrogel is chemically cross-linked by a biocompatible small-molecule reinforcing agent.

Prior to use, the reinforced constructs may be cultured in suitable media for a length of time to allow for further cell proliferation and growth. The constructs may be cultured for at least one week, 2 weeks, 3 weeks, 4 weeks or even longer. According to a particular embodiment, the constructs are perfused in a perfusion chamber or bioreactor prior to use. Examples of media suitable for culturing the construct include, but are not limited to EGM-2 (Lonza, cat #CC-3162/6), and other DMEM based supplemented medium.

According to some embodiments, perfusion is effected subsequent to contacting the construct with the reinforcing agent according to the respective embodiments. Alternatively, perfusion is effected prior to contacting the construct with the reinforcing agent.

According to some of any of the embodiments described herein, the cellularized engineered construct is such that the biocompatible small-molecule reinforcing agent is chemically interacted with (e.g., covalently bound to) at least 10, at least 20, at least 30, at least 40, at least 50, %, or more, % of respective chemically compatible groups (e.g., free amine groups as described herein) present in the ECM-based hydrogel (before it chemically interacted with the reinforcing agent). That is, the number of free chemically compatible groups (e.g., free amine groups) in the ECM-based hydrogel is lower by at least 10%, at least 20%, at least 30%, at least 40%, at least 50%, at least 60%, or more, compared to the same number of free groups in the same ECM-based hydrogel which was not interacted with the reinforcing agent. Determining the number of free functional groups can be performed by analytical measurements known in the art. An exemplary method of determining the number of free amine groups is described in the Examples section that follows, and utilizes spectroscopic measurements (e.g., absorption measurements) upon reacting the construct with a spectroscopically active agent that binds to the respective free groups.

In some embodiments, the constructs described herein are used for scientific and/or medical research. Suitable scientific and/or medical research includes both in vivo and in vitro research. In further embodiments, the engineered, constructs described herein, are for in vitro research uses including, by way of non-limiting examples, disease modeling, drug discovery, and drug screening.

The retinal constructs of the present invention are suitable for implantation and may be used for treating any disorder or condition associated with retinal degeneration. In various embodiments, the constructs are suitable for implantation in any vertebrate subject in need of, for example,

In some embodiments, the shape is selected to mimic the natural shape of the eye. In some embodiments, the size of engineered constructs, including those bioprinted, change over time. In further embodiments, a bioprinted construct shrinks or contracts after bioprinting due to, for example, cell migration, cell death, cell-adhesion-mediated contraction, or other forms of shrinkage. In other embodiments, a bioprinted tissue or organs grows or expands after bioprinting due to, for example, cell migration, cell growth and proliferation, cell maturation, or other forms of expansion.

Eye conditions for which the constructs may serve as therapeutics include, but are not limited to retinal diseases or disorders generally associated with retinal dysfunction, retinal injury, and/or loss of photoreceptor function. A non-limiting list of conditions which may be treated in accordance with the invention comprises retinitis pigmentosa, lebers congenital amaurosis, hereditary or acquired macular degeneration, age related macular degeneration (AMD), dry AMD, Best disease, retinal detachment, gyrate atrophy, choroideremia, pattern dystrophy as well as other dystrophies of the RPE, Stargardt disease, RPE and retinal damage due to damage caused by any one of photic, laser, inflammatory, infectious, radiation, neo vascular or traumatic injury.

Subjects which may be treated include primate (including humans), canine, feline, ungulate (e.g., equine, bovine, swine (e.g., pig)), avian, and other subjects. Humans and non-human animals having commercial importance (e.g., livestock and domesticated animals) are of particular interest. Exemplary mammals which may be treated include, canines; felines; equines; bovines; ovines; rodentia, etc. and primates, particularly humans. Non-human animal models, particularly mammals, e.g., primate, murine, lagomorpha, etc. may be used for experimental investigations.

The retinal constructs generated as described herein may be implanted to various target sites within a subject's eye. In accordance with one embodiment, the implantation of the retinal constructs is to the subretinal space of the eye. In addition, dependent upon migratory ability and/or positive paracrine effects of the cells, transplantation into additional ocular compartments can be considered including the vitreal space, inner or outer retina, the retinal periphery and within the choroids.

The implantation may be performed by various techniques known in the art. Methods for performing retinal cell transplants are described in, for example, U.S. Pat. Nos. 5,962,027, 6,045,791, and 5,941,250 and in Eye Graefes Arch Clin Exp Opthalmol March 1997; 235 (3): 149-58; Biochem Biophys Res Commun Feb. 24, 2000; 268 (3): 842-6; Opthalmic Surg February 1991; 22 (2): 102-8. Methods for performing corneal transplants are described in, for example, U.S. Pat. No. 5,755,785, and in Eye 1995; 9 (Pt 6 Su): 6-12; Curr Opin Opthalmol August 1992; 3 (4): 473-81; Ophthalmic Surg Lasers April 1998; 29 (4): 305-8; Ophthalmology April 2000; 107 (4): 719-24; and Jpn J Ophthalmol November-December 1999; 43 (6): 502-8. If mainly paracrine effects are to be utilized, cells may also be delivered and maintained in the eye encapsulated within a semi-permeable container, which will also decrease exposure of the cells to the host immune system (Neurotech USA CNTF delivery system; PNAS Mar. 7, 2006 vol. 103 (10) 3896-3901).

The effectiveness of treatment may be assessed by different measures of visual and ocular function and structure, including, among others, best corrected visual acuity (BCVA), retinal sensitivity to light as measured by perimetry or microperimetry in the dark and light-adapted states, full-field, multi-focal, focal or pattern electroretinography ERG), contrast sensitivity, reading speed, color vision, clinical biomicroscopic examination, fundus photography, optical coherence tomography (OCT), fundus auto-fluorescence (FAF), infrared and multicolor imaging, fluorescein or ICG angiography, adoptive optics and additional means used to evaluate visual function and ocular structure.

The subject may be administered corticosteroids prior to or concurrently with the administration of the constructs, such as prednisolone or methylprednisolone, Predforte.

Immunosuppressive drugs may be administered to the subject prior to, concurrently with and/or following treatment.

The immunosuppressive drug may belong to the following classes:
Glucocorticoids, Cytostatics (e.g., alkylating agent or antimetabolite), antibodies (polyclonal or monoclonal), drugs acting on immunophls (e.g., ciclosporin, Tacrolimus or Sirolimus). Additional drugs include interferons, opiods, TNF binding proteins, mycophenolate and small biological agents.
Examples of immunosuppressive drugs include: mesenchymal stem cells, anti-lymphocyte globulin (ALG) polyclonal antibody, anti-thymocyte globulin (ATG) polyclonal antibody, azathioprine, BAS1 L1X1MAB® (anti-I L-2Ra receptor antibody), cyclosporin (cyclosporin A), DACLIZUMAB® (anti-I L-2Ra receptor antibody), everolimus, mycophenolic acid, RITUXIMAB® (anti-CD20 antibody), sirolimus, tacrolimus, Tacrolimus and or Mycophenolate mofetil.

Antibiotics may be administered to the subject prior to, concurrently with and/or following treatment. Examples of antibiotics include Oflox, Gentamicin, Chloramphenicol, Tobrex, Vigamox or any other topical antibiotic preparation authorized for ocular use.

It is expected that during the life of a patent maturing from this application many relevant printing technologies will be developed and the scope of the term printing is intended to include all such new technologies a priori.

As used herein the term “about” refers to +10% or +5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc, as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

For any of the embodiments described herein, a reinforcing agent as described herein may be in a form of a salt, for example, a pharmaceutically acceptable salt.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

Further, a reinforcing agent as described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The reinforcing agent described herein can be used as polymorphs and the present embodiments further encompass any isomorph of the compounds and any combination thereof.

The reinforcing agent described herein encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an(S) configuration and any combination, and compounds according to some embodiments of the present invention, can have any their chiral centers exhibit an (R) or an(S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### MATERIALS AND METHODS

Extracellular matrix hydrogel production: The omentum ECM hydrogel was produced as described before²³ and kept at 4° C. in a liquid stage until printing. Omental tissue (Kibutz Lahav, Israel) was agitated for 1 h in hypotonic buffer (10 mM Tris-HCl, 5 mM ethylenediaminetetraacetic acid (EDTA) and 1 μM phenylmethanesulfonyl-fluoride at pH 8.0. The tissue was then subjected to three cycles of freeze-thaw (−80° C.-37° C.) using the same buffer. After the last cycle, the tissue was gradually dehydrated by washing it once with 70% ethanol for 30 min and three times in 100% ethanol for 30 min each. Polar lipids of the tissue were then extracted by three 30 min washes of 100% acetone. Subsequently, the a-polar lipids were extracted by three incubations in a 60:40 hexane:acetone solution (8 h each). Then, the defatted tissue was gradually rehydrated and subjected to 0.25% Trypsin-EDTA (solution B, Biological Industries) degradation overnight at room temperature (RT). The tissue was then thoroughly washed with PBS, followed by incubation with 1.5 M NaCl for 24 h (including three solution changes). After 24 h the tissue was washed in 50×10³ m Tris (pH 8.0), 1% triton-X100 (Sigma) solution for 1 h. The decellularized tissue was washed in PBS followed DDW and then frozen (−20° C.) and lyophilized. After lyophilization, the decellularized omentum was ground into a coarse powder using a Wiley Mini-Mill and then frozen until further use.

Pre-printing, the decellularized omentum ECM (dECM) powder was enzymatically digested by adding 1 mg/mL of porcine pepsin (Sigma, 3200-4500 units mg-1 protein) in 0.1 M HCl. The final concentration of dECM was 1% (w/v). The dECM was digested for 64-72 h at RT under constant stirring until the liquid was homogenous with no visible particles. Subsequently, the salt concentration was adjusted to physiological levels using DMEM-F12 and to terminate pepsin activity, the pH was raised to 7.2-7.4 using 5 M NaOH.

Sacrificial material preparation (support medium): The generation of the sacrificial material was done as previously described²⁰, an aqueous solution containing 0.32% (w/v) sodium alginate (PROTANAL LF 200 FTS, a generous gift from FMC BioPolymer), 0.25% (w/v) pre-treated XG, 37.5 mM sodium chloride, and 9.56 mM calcium carbonate (in suspension, Sigma-Aldrich) were prepared. While constantly stirred, the mixture was supplemented with freshly prepared, pre-dissolved D-(+)-Gluconic acid δ-lactone (GDL, Sigma-Aldrich) to reach a final concentration of 19.15 mM. This results in a slow decrease in the pH, gradual solubilization of the calcium carbonate, and liberation of the calcium ion that crosslinks the alginate. When the solution's viscosity is increased to a level that prevents precipitation of the calcium carbonate, the stirring was stopped and the mixture was incubated at RT for 24 h. DDW was added at 4 times the volume of the resulted hydrogel, followed by homogenization at 25000 RPM for 2 min (HOG-020 homogenizer with GEN-2000 generator probe, MRC ltd, Israel). The homogenate was incubated for 24 h at 4° C., and then centrifuged at 15800 g for 20 min. The pellet was washed by resuspension (vigorous vortexing) in DDW, recentrifuged, and resuspended in Dulbecco's modified Eagle medium (DMEM)/F12 (HAM) 1:1 culture media (Biological Industries, Israel) and centrifuged again, after which the supernatant was discarded. Next, 1.1% (w/v) XG in DDW was added to the pellet at a 1:20 volume ratio to reach a final concentration of ˜0.05% (w/v), followed by vigorous vortexing to homogenize the mixture. The mixture was then incubated for 3-4 days or 6-8 days at RT or 4° C., respectively. After this period, the mixture could be used immediately to support printing, or, alternatively, stored at room temperature or 4° C. for later use.

Sacrificial material extraction: In order to extract the sacrificial material after printing, the printed disk was incubated at 37° C. for 30 min to cure the bioink. As a next step, alginate lyase was added to the growth medium (Sigma-Aldrich, 1 U ml⁻¹) and the disk incubated at 37° C. until the digested sacrificial material became liquid.

Rheological Properties: Rheological measurements were taken using Discovery HR-3 hybrid Rheometer (TA Instruments, DE) with 20 mm diameter parallel plate geometry with a Peltier plate to maintain the sample temperature. The samples were loaded at a temperature of 4° C., which was then raised to 37° C. to induce gelation; during which the oscillatory moduli of samples were monitored at a fixed frequency of 1 Hz and a strain of 1%.

Cell culture: ARPE-19 cells were cultured in Dulbecco's Modified Eagle Medium (DMEM): Nutrient Mixture F-12 (Ham's) (1:1), with Sodium Bicarbonate 1.2 gm/l, with Hepes 15 mM, with Sodium Pyruvate 55 mg/L, supplemented with 10% FBS, 1% L-Glutamine, 1% Penicillin-Streptomycin Solution (all from Biological Industries). The cell medium was replaced twice a week. Primary human umbilical vein endothelial cells (HUVECs) (Lonza) were maintained in EGM2 (Lonza) supplemented according to the manufacturer instructions. Immortalized GFP labeled HUVECs were cultured in DMEM supplemented with 10% FBS, 1% L-Glutamine, 1% Penicillin-Streptomycin, the cell medium was replaced every other day. An early passage (12) of 661 W photoreceptors cell line was a kind gift provided by Prof. Muyyad R. Al-Ubaidi, the cells were grown in DMEM supplemented with 10% FBS, 1% L-Glutamine, 1% Penicillin and Streptomycin at 37° C. in 5% CO₂. hES-RPE cells were a kind gift from Prof. Benjamin Reubinoff and Dr. Maria Idelson²⁴ the cells were grown in knockout DMEM supplemented with 14% knockout serum, 1% penicillin and streptomycin, 1% Alpha MEM, 2 M nicotinamide at 37° C. in 5% CO₂. The cell medium was replaced every other day. hiPSC-derived endothelial cells were differentiated as previously described²⁵. Briefly, human iPSCs were dissociated on day 0 with Accutase and replated on Matrigel™, diluted to 50 μg/mL in DMEM/F12, coated plates. Cells were seeded at a density of 47,000 cells/cm² and maintained in NutriStem™ medium containing 1% Penicillin/Streptomycin and 10 μM Y-27632 ROCKi. On day 1, the medium was replaced with mesoderm induction medium containing a 1:1 (v/v) mix of Neurobasal and DMEM/F12 supplemented with L-Glu, N2 and B27 minus retinoic acid with 25 ng/mL BMP4 and 8 μM CHIR99021. The media was not changed for 3 days to induce a mesoderm state. On day 4, the medium was changed to EC induction medium consisting of StemPro-34 SFM medium supplemented with 200 ng/mL VEGF165 and 2 μM forskolin. The EC induction medium was changed daily. On day 7, the cells were dissociated with Accutase and magnetic-activated cell sorting (MACS) was used to separate for CD31+CD144+ cells. The sorting was performed using a manual MACS® magnetic separator and magnetic beads conjugated antibodies. The CD31+/CD144+ cells were seeded onto cell culture treated flasks and cultured in EGM-2 supplemented with 20 μM SB431542. Media was replaced every other day. All cells were passaged using 0.05% trypsin (Biological Industries). The printed structure with ARPE-19, HUVECs, and 661w was cultured in EGM2; media was replaced every other day. The printed structure with hES-RPE and hiPSC-derived endothelial cells was cultured in the same media as hES-RPE. Media was replaced every other day.

Perfusion culture: A perfusion chamber was designed using an open-source design²⁶ and printed with a MAX X43 DLP printer (ASIGA). The implant was printed directly in the perfusion chamber and connected to a multichannel peristaltic pump (Ismatec, ISM93D) with a 27 G needle and Ismatec pump tubing (2-Stop, PharMed® BPT, 0574-95723-12). Media was perfused at a rate of 104 μL/h.

Immunofluorescence staining: Samples were fixated after the culture medium was taken out, and the sample was washed with cold PBS twice. Cells were fixated using 4% PFA in PBS for 20 minutes at RT. The samples were then washed 3 times with PBS. The samples were permeabilized with 0.1%×100 Triton in PBS for 5 minutes. The samples were then washed 3 times with PBS. Blocking solution (2% BSA in PBS) was added to the samples for 1 hour at RT. Primary antibodies were diluted in blocking solution according to table 1 and added to the samples for 1 hour at RT or at 4° C. over night. Secondary antibodies were incubated for 1.5 hours: goat anti rabbit Alexa 488 (Abcam, ab150077, 1:250), goat anti mouse Alexa 647 (Abcam, ab150119, 1:250), goat anti mouse Alexa 555 (Abcam, ab150118). Phalloidin staining was done by adding phalloidin conjugated to iFluor 647 (Abcam, ab176759, 1:1000) or phalloidin conjugated to iFluor 555 (Abcam, ab176756, 1:1000). For nuclei detection, samples were incubated for 5 min with Hoechst 33258 (1:20; Sigma) Images were taken using confocal microscopy (Nikon Eclipse Ni) or an inverted fluorescence microscope (Nikon Eclipse TI).

TABLE 1
Primary antibodies used for immunofluorescence staining

| | Working | Catalog | |
|---|---|---|---|
| Name | dilution | number | Manufacturer |
| Rabbit anti-PAX6 | 1:150 | ab195045 | Abcam |
| Mouse anti-BEST1 | 1:150 | ab2182 | Abcam |
| Rabbit anti-OTX1/2 | 1:150 | ab21990 | Abcam |
| Mouse anti-RPE65 | 1:100 | NB100-355 | Novus |
| Rabbit anti-ZO1 | 1:200 | CST-13663S | Cell signaling |
| Mouse anti-CD31 | 1:250 | P8590 | Sigma |
| Chicken anti-nestin | 1:2000 | ab134017 | Abcam |
| Rabbit anti-collagen IV | 1:500 | ab6586 | Abcam |
| Rabbit anti-laminin | 1:50 | ab11575 | Abcam |
| Mouse anti-collagen I | 1:4000 | MA126771 | Invitrogen |
| Rabbit ant-Phospho-Ezrin | 1:200 | 3141 | Cell signaling |
| Mouse anti-beta-catenin | 1:200 | sc-7963 | Santa Cruz |

Phagocytosis assay: ARPE-19 or hRPE cells were grown until confluence on dECM coated 13-mm diameter glass cover slides in a 24-well plate. 1 μm carboxylate-modified polystyrene, fluorescent yellow-green beads (Sigma) were prepared by 3 washes with sterile PBS to remove Sodium Azide. The cells were incubated for 16 hours at 37° C. in medium containing different concentrations of the beads. The samples were washed 3 times with PBS, fixated for 20 minutes with 4% PFA and stained with Alexa-Fluor-647-conjugated Phalloidin (Sigma) For visualization of F-actin. The fluorescence signal was observed with a laser-scanning confocal microscope and the amount of phagocytotic cells was counted manually. Statistical significance was determined by an unpaired t-test.

Calcium imaging: Fluo-4-AM was prepared by adding 45 μL DMSO and 45 μL Pluronic-DMSO to a 50 μg vial of Fluo-4-AM.Fluo-4-AM was diluted with HBSS in a ratio of 1:50 and the cells were incubated for 1 hour in 37° C. The cells were washed for 30 minutes in HBSS in 37° C. The sample was placed in an inverted microscope on a heating plate adjusted to 37° C. Fluo-4-AM emission is at 506 nm and excitation is at 494 nM. Images were acquired at a rate of 3.15 Hz. 10 seconds After starting the recording 100 μM ATP were added to the medium. The data was analyzed using ImageJ. TEM: TEM Samples were fixated in 2.5% Glutaraldehyde in PBS over night at 4° C. After several washes in PBS, the samples were post fixed in 1% OsO4 in PBS for 2 h at 4° C. Dehydration was carried out in graded ethanol followed by embedding in Glycid ether. Thin sections were mounted on Formvar/Carbon coated grids, stained with uranyl acetate and lead citrate and examined in Jeol 1400-Plus transmission electron microscope (Jeol, Japan). Images were captured using SIS Megaview III and iTEM the Tem imaging platform (Olympus).SEM: Samples for SEM were fixed with 2.5% glutaraldehyde (24 h, at 40C), followed by a graded incubation series in ethanol-water solutions (50-100% (v/v)). Then, the samples were critical point dried, sputter-coated with gold in a Polaron E 5100 coating apparatus (Quorum technologies, Lewis, UK) and observed under JSM-840A SEM (JEOL, Tokyo, Japan).

Trans-epithelial electrical resistance (TEER): The barrier properties of the ARPE-19 cells cultured on dECM hydrogel were evaluated with TEER measurements. ARPE-19 cells were seeded on 24-well inserts (Greiner) coated with dECM hydrogel at a density of 1.7×10⁵ cells/cm² and cultured for 4 weeks. TEER was measured with the Millicell ERS-2 Voltohmmeter (Merck Millipore). TEER values (Ω×cm²) were calculated by subtracting the TEER of a similarly coated insert without cells, and by multiplying the result by the surface area. TEER values were obtained 3 parallel samples, and 3 technical replicates.

### RESULTS

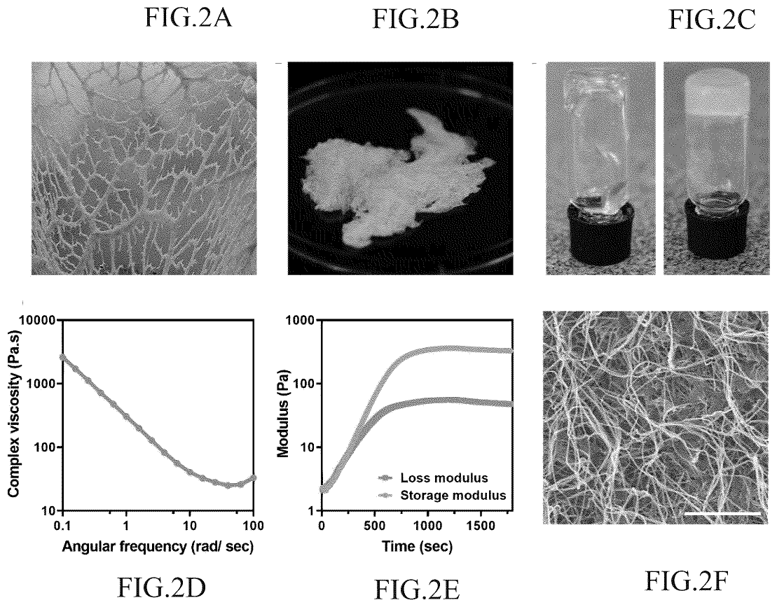

To fabricate the ECM hydrogel, an omental ECM was used. The omentum is a fatty tissue containing blood vessels and glycosaminoglycans, and has remarkable regenerative capabilities¹⁶. This tissue can be taken from patients by a relatively easy, minimally invasive procedure, to generate a personalized biomaterial²⁷. As a proof of concept, omental tissues were obtained from porcine and subjected to a decellularization procedure, preserving the ECM proteins (FIGS. 2A-B). The decellularized tissue was then processed into a thermoresponsive weak, liquid-like hydrogel at room temperature that became a viscous, physically crosslinked hydrogel under physiological conditions (FIGS. 2C-E). These properties enabled the hydrogel to be manipulated and structured prior to gelation. Additionally, SEM images revealed that the hydrogel was composed of ECM nanofibers that may encapsulate cells and promote multiple anchoring sites for cell adhesion (FIG. 2F).

The present inventors sought to exploit an omentum-based hydrogel for printing the choroid, which is the blood vessel layer supporting the outer retina. To generate a blood vessel with a 300 μm diameter and a capillary bed, endothelial cells were printed at room temperature in a sacrificial material which was surrounded by a printed ECM hydrogel (FIG. 3A). During the gelation of the ECM hydrogel, the sacrificial material supported it, retaining its structure. As the sacrificial material properties do not provide cell anchors, the cells adhered to the surrounding ECM hydrogel. After complete gelation at 37° C., the sacrificial material was enzymatically digested and removed, leaving endothelial cells in a vessel-like structure with an internal diameter of approximately 300 μm (FIG. 3B). To ensure sufficient nutrients and oxygen transfer from the printed structure, the blood vessel network was connected to a peristaltic pump, and proper perfusion was demonstrated (FIG. 3C). The combination of convection through the vessel by perfusion, and diffusion from the vessels and into the hydrogel, ensures a proper mass transfer throughout the fabricated structure²⁰. Additionally, to facilitate the formation of a capillary bed, two blood vessels could be printed in parallel (FIG. 3D) to promote endothelial cell migration in the ECM hydrogel (FIG. 3E). Finally, to ensure that the endothelial cells indeed lined up the printed lumen, confocal microscope imaging was performed (FIG. 3F).

Proper interaction between the RPE cells and photoreceptors requires initial maturation of the RPE in a polarized monolayer²⁸. Therefore, a step-by-step process is needed, where the RPE layer is first assembled and matured before continuing to engineer the photoreceptor layer. The RPE layer was deposited dropwise on top of the printed choroid and allowed to self-organize and mature for 7 days. Next, the present inventors assessed the morphology of the RPE monolayer by analyzing the formation of tight junctions between the cells. Tight junctions play an important role in the barrier function of the RPE cells, helping to maintain a proper balance of nutrients and waste products within the retina²⁹. As shown, immunostaining for the tight junction marker ZO1 confirmed RPE monolayer junctional maturity (FIG. 4A). Moreover, co-culturing the cells with the printed blood vessels, promoted features characteristic of RPE, including defined cell borders, an overall ‘cobblestone’ appearance³⁰, and expression of key RPE markers, such as OTX½, PAX6, BEST1 and RPE6531 (FIGS. 4B-E). The interface between the printed blood vessels and the RPE layer was demonstrated by co-staining the construct for CD31 and PAX6 (FIG. 4F). The basolateral localization of BEST1 demonstrated the polarity of the RPE monolayer (FIG. 4G), which is essential for nutrient transport and waste disposal between the photoreceptors and the choroid. If the polarity of the RPE cells is disrupted, it can lead to a misfunctioning retina³².

Normally in the healthy functioning retina, phototoxic damage affects the outer segment of photoreceptors, and the cells undergo a continuous turnover, where the underlying RPE monolayer phagocytes the tips of the outer segment³³. Therefore, to assess the function of the engineered RPE layer, the present inventors next incubated the cells with fluorescent latex beads (d=1 μm), and the ability of the engineered RPE layer to perform phagocytosis was demonstrated (FIG. 4H). Moreover, several key RPE functions, such as the transport of nutrients and waste products across it, the maintenance of the blood-retinal barrier, and the regulation of cell growth and differentiation, are regulated by calcium ions³⁴.

Calcium signaling in RPE is activated by light-induced increases in adenosine triphosphate (ATP) in the subretinal space, affecting, for instance, the regulation of the hydration and chemical composition of the subretinal space and the adhesion of the retina³⁵. As shown, the engineered RPE layer reacted to ATP with the activation of calcium signaling pathways (FIG. 4I). The addition of ATP immediately increased intracellular calcium levels in a significant manner, followed by a slow decrease to the baseline. Finally, to demonstrate that the RPE layer formed a barrier when cultured on top of the ECM hydrogel containing the blood vessels, trans-epithelial electrical resistance (TEER) measurements were performed during 4-weeks of cultivation. TEER value is a physiological index used to evaluate the permeability of paracellular pathways by measuring the transport rate of ions or macromolecules. As shown, TEER significantly increased after 3 weeks of culture and was maintained until 4 weeks at 21.09±0.67 Ω2×cm² (FIG. 4J). A similar TEER value was measured for this cell line on different coatings³⁶. After engineering the choriocapillaris-RPE complex using cell lines, a more clinically relevant complex, composed of pluripotent stem cell-derived RPEs and ECs, was printed and cultured for 14 days. Immunostaining confirmed the maturity and polarity of the RPE layer with cobblestone morphology (FIG. 4K), and Ezrin and beta-catenin expression in a polar manner (FIG. 4L). The ezrin/radixin/moesin family of proteins localizes to microvilli of epithelia, where it acts as a bridge between actin filaments and plasma membrane proteins³⁷. Beta-catenin is a protein with dual functions involved in both gene transcription as the downstream effector of Wnt signaling and as a cell-cell adhesion protein. RPE cells bind to the cytoplasmic domain of cadherin through beta-catenin that is found at the basal plasma membrane³⁸. Co-culturing the hES-RPE cells with the printed ECs did not result in the loss of typical RPE phenotype, as demonstrated by the expression of key RPE markers, such as OTX½, PAX6, BEST1, and RPE65 (FIGS. 4M-N).

After 7 days, when the RPE cells had already formed a mature monolayer and a close interface with the underneath blood vessel network, photoreceptor cells (661w cells) were seeded on top of the construct and cultivated for up to 94 days. Immunostaining confirmed the maturity of the RPE layer that expressed BEST1 and the presence of nestin-expressing photoreceptors (FIG. 5A). Furthermore, a cross-section image of the construct revealed the assembly of the photoreceptors on top of the RPE layer (FIG. 5B). Such localization of the cells is crucial for the viability and maintenance of the photoreceptors as they are supported by the RPE layer. Furthermore, cultivation of the 3-layer retina-like structure for more than 100 days revealed key ultrastructural features of RPE (FIG. 5C), including pigmentation (black arrow), microvilli (blue arrow), and desmosomes (green arrow), which are important for RPE cell function³⁹. For example, melanosomes are pigment particles within the RPE cells that give them their characteristic dark color. These pigment granules help to absorb light, reducing any stray light that may interfere with vision within the retina. Microvilli are small, finger-like protrusions on the surface of RPE cells that help to increase the surface area available for the exchange of nutrients, waste products, and signaling molecules between the RPE and photoreceptors. Desmosomes are special types of cell-cell junctions that help to anchor RPE cells to each other and to the photoreceptors. By providing structural support and stability to the RPE layer, these junctions help to prevent damage to the photoreceptors during eye movements. Additionally, desmosomes regulate the exchange of materials between RPE cells and photoreceptors, helping to maintain their health and function^{40,41}. The existence of these features within the supporting RPE layer may provide another evidence for its maturation^{32,33}.

Bruch's membrane is a thin, extracellular matrix layer that separates the retina from the choroid in the eye. It is composed of collagen I and IV, laminin, and other proteins and is an important structural element in the eye. The existence of this structure helps to maintain the shape of the retina and provides a substrate for the attachment of the RPE cells⁴². Therefore, the present inventors next sought to examine the existence of this ECM layer within the 3-layer structure and its interaction with the cells. Immunostaining for ECM proteins of the Bruch's membrane confirmed their existence between the RPE and the printed choroid (FIGS. 5D-F). As shown, the barrier between the RPE and blood vessel layers contained collagen I and IV (FIGS. 5D-E). Another key component of Bruch's membrane is laminin, which crosslinks the collagen fibers, structuring them as a 2D mesh⁴³. As shown, the formed Bruch's membrane contained laminin, expressed in the basolateral side of the RPE monolayer (FIG. 5F). Overall, the detection of these ECM proteins indicates that our 3D model enabled the creation of Bruch's membrane-like structures in vitro that enable close interactions between RPE and choriocapillaris. Such structure can replace the damaged Bruch's membrane in the patient, as well as support the cells of the retina and provide them with a proper environment for survival and function.

### Example 2

### REINFORCEMENT OF HYDROGELS

### Materials and Methods

Synthesis of SOx: Oxidized sucrose (SOx) was prepared following the protocol established by Liu et al., [Macromol. Mater. Eng. 2015, 300, 414]. Briefly, a 33 mM solution of D-sucrose (Bio-Labs) in acetate buffer (pH=5) was stirred at room temperature while a 0.2 M NaIO4 (Fischer Chemical) solution was added dropwise at the rate of 1 mL min-1 by a syringe pump (NE-1000; New Era Pump Systems). The entire solution was kept in the dark and allowed to stir overnight. A 1 M BaCl₂ (Acros Organics) solution was added dropwise until further addition of the BaCl₂ resulted in no further precipitation of iodine salts. The mixture was filtered to remove precipitates, and a 0.1 M NaOH solution was carefully added until the solution reached a pH of approximately 7.4. The mixture was again filtered to remove the barium salts, then lyophilized and stored at −20° C. until use.

### Preparation of Decellularized Omentum Hydrogel: as in Example 1.

Collagen Dissociation with SDS: Hydrogel samples (n=3) were submerged in a fresh 1% solution of Sodium dodecyl sulfate (Sigma) in PBS. The absorbance of light of wavelength 600 nm (Infinite M200 Pro; Tecan, Switzerland) was assessed every 15 minutes at several different locations within the well. The highest absorbance was taken as the reading.

### Compressive Modulus:

The compressive modulus measurements were performed using a Discovery HR-3 Hybrid Rheometer (TA Instruments, DE) with a 20 mm diameter parallel plate geometry and a Peltier plate to maintain the sample temperature. Bulk modulus tests were performed by compressing the samples at a fixed rate of 100 μm s⁻¹.

### RESULTS

The mechanical properties of an exemplary native ECM-based hydrogel (derived from omentum) and of cross-linked hydrogels were assessed by rheological measurements. Shear thinning measurements were conducted at 37° C. Data are presented as mean±SD, n=3. P-values were calculated using a Comparison of Fits test with an Extra Sum of Squares F-test.

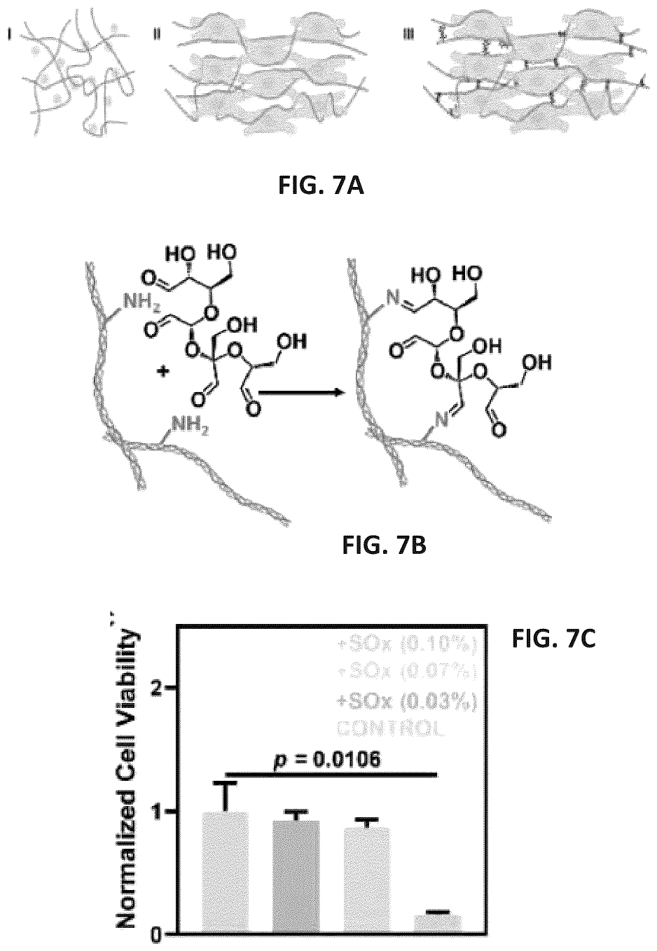

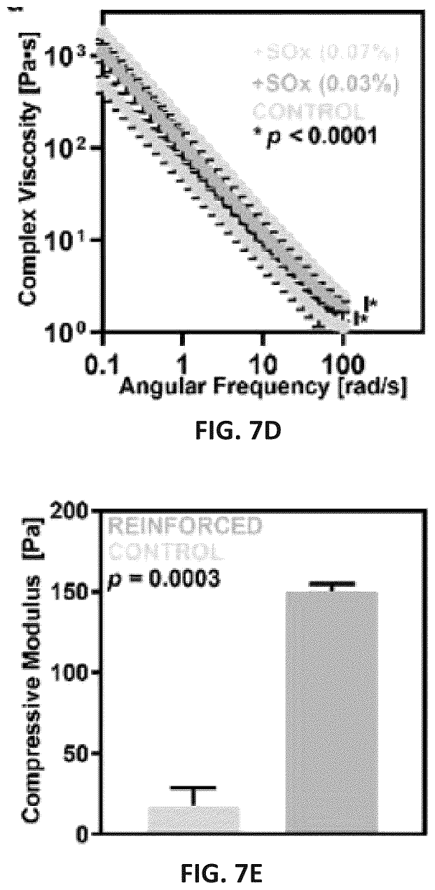

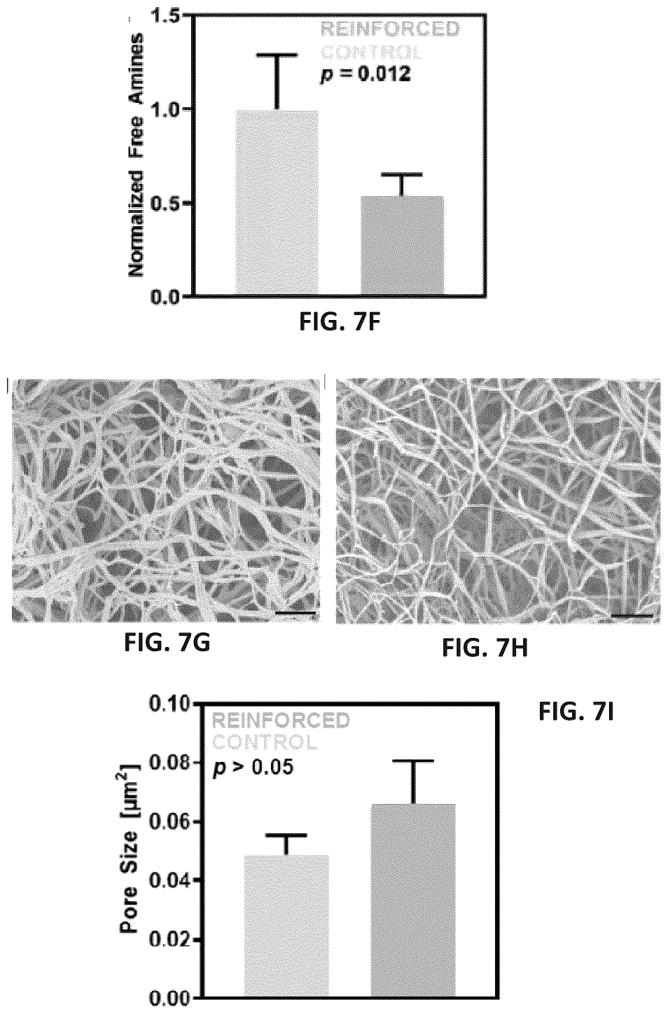

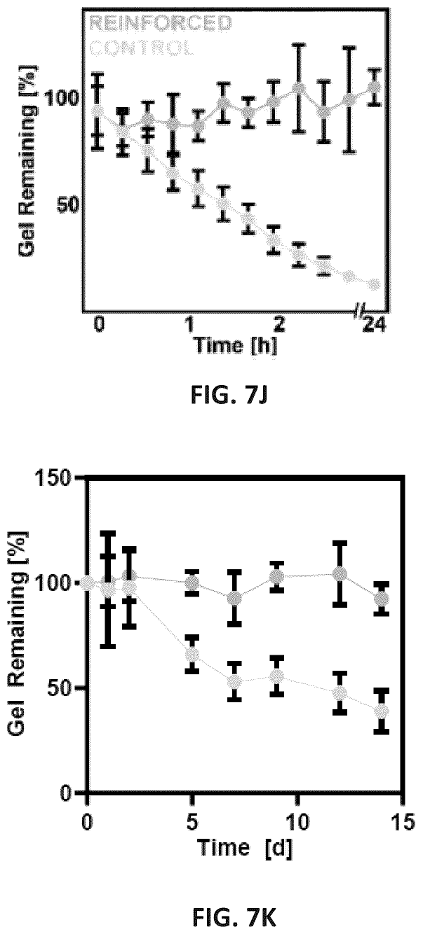

The data are presented in FIGS. 7A-K and show that at all frequencies, the reinforced hydrogels demonstrated superior mechanical properties. While a large increase in gel viscosity was observed between the control gel and a sample cross-linked with 0.03% SOx, a smaller increase was seen when increasing the SOx concentration to 0.07%. Therefore, 0.03% SOx was used in further studies.

Alongside the rheological testing, the bulk mechanical properties of the hydrogel were also assessed. Samples were compressed, and the linear elastic modulus was calculated. Data are presented as mean±SD, n=3. P-values were calculated using a one-tailed, unpaired, parametric t-test assuming that all samples have the same distribution.

The data are presented in FIG. 7E, revealing significant reinforcement of the hydrogel.

To determine the ability of SOx to react with an ECM-based hydrogel, the concentration of amine groups present in the ECM before and after exposure to the Sox was assessed. The data are presented in FIG. 7F. As can be seen, the absorbance of a solution of ninhydrin that was allowed to react with the hydrogel was normalized to the control value and showed that 46% of the amines had reacted with the Sox cross-linker. Data are presented as mean±SD, n=4. All values were normalized to the average value of the control group. P-values were calculated using a one-tailed, unpaired, parametric t-test assuming that all samples have the same distribution.

The morphology of ECM-based hydrogel before and after exposure to Sox was tested and the obtained data is shown is FIGS. 7G-I.

High-resolution imaging of non-crosslinked ECM hydrogel (before exposure to Sox) and of cross-linked ECM hydrogel (after exposure to Sox) are presented in FIGS. 7G and 7H, respectively (Scale bar=1 micrometer), and revealed that the reinforcement with SOx proceeded without altering the internal morphology.

In FIG. 7I the data shown in the obtained images was used to evaluate the pore size, and further show that the reinforcement protocol did not alter the overall mesh formation of the hydrogel. The size of the holes in the mesh were calculated before and after crosslinking, and no difference was observed between the samples. Data in FIG. 7I is presented as mean±SD, n=3, and each image had at least 180 pores whose area was determined using ImageJ. P-values were calculated using a two-tailed, unpaired, parametric t-test assuming that all samples have the same distribution.)

These results suggest that the SOx molecules do not form new adhesion points between the hydrogel fibers, but rather chemically cross-link the existing entanglement points that had already been cross-linked physically. Additionally, the fact that the fibers did not compact during reinforcement suggests that SOx is able to penetrate deep into the tissue and provide uniform reinforcement. It is to be noted that this stands in contrast to most chemical cross-linkers, which cause the polymer network to become more tightly packed, slowing the diffusion rate into the gel and leading to non-uniform cross-linking [see, for example, Wu et al., J. Phys. Chem. B 2009, 113, 3512].

Because cells are uniformly distributed throughout the entire structure, this represents a significant advantage of using SOx as a reinforcer.

To further support these findings, both the control and reinforced hydrogels were exposed to sodium dodecyl sulfate (SDS). SDS is a detergent that binds to peptide chains, imparts a negative charge, and causes them to repel one another [Lambin and Rochu, J. M. Fine, Anal. Biochem. 1976, 74, 567; Reynolds and Tanford, J. Biol. Chem. 1970, 245, 5161].

Because the addition of SDS leads to electrostatic repulsion, weak, physically entangled gels disintegrate in its presence while more strongly, chemically cross-linked gels swell but do not break.

The data obtained for hydrogel disintegration in SDS is shown in FIG. 7J. Data presented as mean±SD, n=3. All values normalized to the average reading across all six samples at t=0. P-values were calculated using a two-tailed, unpaired, parametric t-test assuming that all samples have the same distribution). As compared to the pristine non-reinforced hydrogel, which rapidly disintegrated overnight, the reinforced hydrogel remained intact and only swelled due to the chemical cross-linking that had occurred.

Degradation studies were also performed using the biologically relevant enzyme collagenase, and the obtained data is shown in FIG. 7K. Data presented as mean±SD, n=3. Each sample was individually normalized to its initial value. P-values were calculated using a two-tailed, unpaired, parametric t-test assuming that all samples have the same distribution.

As shown in FIG. 7K, when non-reinforced hydrogels were exposed to collagenase, 61% rapidly degraded, with only 39% of the gel remaining after two weeks. After reinforcement, however, there was no significant degradation within this time period.

Both of the degradation studies therefore support the observation that SOx provides a uniform reinforcement throughout the hydrogel and does not merely create a cross-linked outer shell.

In addition, any priority document(s) of this application is/are hereby incorporated herein by reference in its/their entirety.

### REFERENCES

1. Nowak, J. Z. Age-related macular degeneration (AMD): Pathogenesis and therapy. Pharmacological Reports 58, 353-363 (2006).
2. Schmitz-Valckenberg, S., Fleckenstein, M., Göbel, A. P., Hohman, T. C. & Holz, F. G. Optical Coherence Tomography and Autofluorescence Findings in Areas with Geographic Atrophy Due to Age-Related Macular Degeneration. Invest Ophthalmol Vis Sci 52, 1-6 (2011).
3. Cruz-Pimentel, M. & Wu, L. Complement Inhibitors for Advanced Dry Age-Related Macular Degeneration (Geographic Atrophy): Some Light at the End of the Tunnel? J Clin Med 12, 5131 (2023).
4. Bhutto, I. & Lutty, G. Understanding age-related macular degeneration (AMD): Relationships between the photoreceptor/retinal pigment epithelium/Bruch's membrane/choriocapillaris complex. Mol Aspects Med 33, 295 (2012).
5. Saksens, N. T. M. et al. Macular dystrophies mimicking age-related macular degeneration. Prog Retin Eye Res 39, 23-57 (2014).
6. Shapira, A. & Dvir, T. 3D Tissue and Organ Printing-Hope and Reality. Advanced Science 8, (2021).
7. Rahmati, M. et al. Electrospinning for tissue engineering applications. Prog Mater Sci 117, 100721 (2021).
8. Zhao, P. et al. Fabrication of scaffolds in tissue engineering: A review. Frontiers of Mechanical Engineering 13, 107-119 (2018).
9. Maeda, T., Mandai, M., Sugita, S., Kime, C. & Takahashi, M. Strategies of pluripotent stem cell-based therapy for retinal degeneration: update and challenges. Trends Mol Med (2022) doi: 10.1016/J.MOLMED.2022.03.001.
10. Falkner-Radler, C. I. et al. Human retinal pigment epithelium (RPE) transplantation: Outcome after autologous RPE-choroid sheet and RPE cell-suspension in a randomised clinical study. British Journal of Ophthalmology 95, 370-375 (2011).
11. Hinkle, J. W., Mahmoudzadeh, R. & Kuriyan, A. E. Cell-based therapies for retinal diseases: a review of clinical trials and direct to consumer “cell therapy” clinics. Stem Cell Research & Therapy 2021 12:1 12, 1-9 (2021).
12. Barzelay, A. et al. Adipose-derived mesenchymal stem cells migrate and rescue RPE in the setting of oxidative stress. Stem Cells Int 2018, (2018).
13. Krief, B. et al. Retinal Lineage Therapeutic Specific Effect of Human Orbital and Abdominal Adipose-Derived Mesenchymal Stem Cells. Stem Cells Int 2021, (2021).
14. Sharma, R. et al. Clinical-Grade Stem Cell-Derived Retinal Pigment Epithelium Patch Rescues Retinal Degeneration in Rodents and Pigs. Sci Transl Med 11, (2019).
15. Sharma, R., Bose, D., Maminishkis, A. & Bharti, K. RETINAL PIGMENT EPITHELIUM REPLACEMENT THERAPY FOR AGE-RELATED MACULAR DEGENERATION-ARE WE THERE YET? Annu Rev Pharmacol Toxicol 60, 553 (2020).
16. Wang, L. et al. The effect of clinical-grade retinal pigment epithelium derived from human embryonic stem cells using different transplantation strategies. Protein Cell 10, 455 (2019).
17. Angel H Garced, R. N. & M. Teresa Magone de Quadros Costa, M. D. Autologous Transplantation of Induced Pluripotent Stem Cell-Derived Retinal Pigment Epithelium for Geographic Atrophy Associated With Age-Related Macular Degeneration. ClinicalTrials (dot) gov (2020).
18. Stahl, A. The Diagnosis and Treatment of Age-Related Macular Degeneration. Dtsch Arztebl Int 117, 513 (2020).
19. Fontaine, V., Kinkl, N., Sahel, J., Dreyfus, H. & Hicks, D. Survival of Purified Rat Photoreceptors In Vitro Is Stimulated Directly by Fibroblast Growth Factor-2. The Journal of Neuroscience 18, 9662 (1998).
20. Noor, N. et al. 3D Printing of Personalized Thick and Perfusable Cardiac Patches and Hearts. Advanced Science 6, (2019).
21. Asulin, M., Michael, I., Shapira, A. & Dvir, T. One-Step 3D Printing of Heart Patches with Built-In Electronics for Performance Regulation. Advanced Science 8, 1-8 (2021).
22. Feiner, R. et al. Engineered hybrid cardiac patches with multifunctional electronics for online monitoring and regulation of tissue function. Nat Mater 15, 679 (2016).
23. Edri, R. et al. Personalized Hydrogels for Engineering Diverse Fully Autologous Tissue Implants. Advanced Materials 31, 1-9 (2019).
24. Idelson, M. et al. Directed Differentiation of Human Embryonic Stem Cells into Functional Retinal Pigment Epithelium Cells. Cell Stem Cell 5, 396-408 (2009).
25. Cohen, R., Baruch, E. S., Cabilly, I., Shapira, A. & Dvir, T. Modified ECM-Based Bioink for 3D Printing of Multi-Scale Vascular Networks. Gels 2023, Vol. 9, Page 792 9, 792 (2023).
26. Kinstlinger, I. S. et al. Perfusion and endothelialization of engineered tissues with patterned vascular networks. Nature Protocols 2021 16:6 16, 3089-3113 (2021).
27. Shevach, M. et al. Omentum ECM-based hydrogel as a platform for cardiac cell delivery. Biomedical Materials (Bristol) 10, (2015).
28. Rodriguez-Boulan, E. J. et al. Conditional knockdown of DNA methyltransferase 1 reveals a key role of retinal pigment epithelium integrity in photoreceptor outer segment morphogenesis. Development 140, 1330-1341 (2013).
29. Naylor, A., Hopkins, A., Hudson, N. & Campbell, M. Tight Junctions of the Outer Blood Retina Barrier. Int J Mol Sci 21, (2020).
30. Reynolds, J. D. & Olitsky, S. E. Pediatric retina. Pediatric Retina 1-462 (2011) doi: 10.1007/978-3-642-12041-1.
31. Burke, J. M. Epithelial phenotype and the RPE: Is the answer blowing in the Wnt? doi: 10.1016/j.preteyeres.2008.08.002.
32. Caceres, P. S. & Rodriguez-Boulan, E. Retinal Pigment Epithelium Polarity in Health and Blinding Diseases. Curr Opin Cell Biol 62, 37 (2020).
33. Boulton, M. & Dayhaw-barker, P. The role of the retinal pigment epithelium: Topographical variation and ageing changes. Nature, Eye 15, 384-389 (2001).
34. Karema-Jokinen, V. et al. Crosstalk of protein clearance, inflammasome, and Ca2+ channels in retinal pigment epithelium derived from age-related macular degeneration patients. Journal of Biological Chemistry 299, 104770 (2023).
35. Wimmers, S., Karl, M. O. & Strauss, O. Ion channels in the RPE. Prog Retin Eye Res 26, 263-301 (2007).
36. Kim, J. et al. Development of 3D printed bruch's membrane-mimetic substance for the maturation of retinal pigment epithelial cells. Int J Mol Sci 22, 1-17 (2021).
37. Bonilha, V. L., Finnemann, S. C. & Rodriguez-Boulan, E. Ezrin promotes morphogenesis of apical microvilli and basal infoldings in retinal pigment epithelium. Journal of Cell Biology 147, 1533-1547 (1999).
38. Li, Q., Scott, P. A., Kaplan, H. J., Dean, D. C. & Lu, Q. Novel function of β-catenin in RPE basal plasma membrane. Invest Ophthalmol Vis Sci 60, 1919-1919 (2019).
39. Tarau, I. S., Berlin, A., Curcio, C. A. & Ach, T. The Cytoskeleton of the Retinal Pigment Epithelium: from Normal Aging to Age-Related Macular Degeneration. International Journal of Molecular Sciences 2019, Vol. 20, Page 3578 20, 3578 (2019).
40. Bennis, A. et al. Stem Cell Derived Retinal Pigment Epithelium: The Role of Pigmentation as Maturation Marker and Gene Expression Profile Comparison with Human Endogenous Retinal Pigment Epithelium. Stem Cell Rev Rep 13, 659-669 (2017).
41. Bonilha, V. L. et al. THE RETINAL PIGMENT EPITHELIUM APICAL MICROVILLI AND RETINAL FUNCTION. Adv Exp Med Biol 572, 519 (2006).
42. Booij, J. C., Baas, D. C., Beisekeeva, J., Gorgels, T. G. M. F. & Bergen, A. A. B. The dynamic nature of Bruch's membrane. Progress in Retinal and Eye Research vol. 29 1-18 Preprint at https://doi (dot) org/10.1016/j.preteyeres.2009.08.003 (2010).
43. Kular, J. K., Basu, S. & Sharma, R. I. The extracellular matrix: Structure, composition, age-related differences, tools for analysis and applications for tissue engineering. J Tissue Eng 5, (2014).

What is claimed is:

1. A method for reinforcing a cellularized retinal construct fabricated from:
(i) endothelial cells;
(ii) retinal pigment epithelial (RPE) cells and/or photoreceptors; and
(iii) an extracellular matrix (ECM) hydrogel,
the method comprising:
contacting the cellularized retinal construct with a biocompatible small-molecule reinforcing agent that is capable of chemically interacting with the ECM hydrogel under conditions that maintain viability of the cells, to thereby increase a compressive modulus of the ECM hydrogel by at least 10%.

2. The method of claim 1, wherein said chemically interacting effects cross-linking of the ECM hydrogel.

3. The method of claim 1, wherein said reinforcing agent is a polyaldehyde.

4. The method of claim 1, wherein said reinforcing agent is an oxidized, poly-aldehyde saccharide.

5. The method of claim 1, wherein said contacting is with a culturing medium that comprises said reinforcing agent.

6. The method of claim 5, wherein said reinforcing agent is an oxidized, poly-aldehyde saccharide and wherein a concentration of said reinforcing agent in said medium is less than 0.1% by weight.

7. The method of claim 1, further comprising generating the cellularized retinal construct prior to the contacting by sequentially forming a plurality of layers on a receiving medium, wherein a first of said layers comprises said endothelial cells and a second of said layers comprises said RPE cells or said photoreceptors.

8. The method according to claim 7, wherein said second layer comprises RPE cells and a third layer comprises said photoreceptors.

9. The method of claim 7, further comprising culturing said cellularized retinal construct for at least 3 days following said generating and prior to said contacting.

10. The method of claim 1, wherein said ECM hydrogel is generated from decellularized omentum.

11. A method of generating an engineered cellularized retinal construct comprising:
(a) culturing endothelial cells in the presence of an ECM hydrogel derived from decellularized omental tissue to generate a layer of endothelial cells;
(b) culturing RPE cells on said layer of endothelial cells to generate a layer of RPE cells; and
(c) culturing photoreceptor cells on said layer of RPE cells, thereby generating the cellularized retinal construct.

12. The method according to claim 11, further comprising contacting the retinal construct with a biocompatible small-molecule reinforcing agent that is capable of chemically interacting with the ECM hydrogel under conditions that maintain viability of the cells, to thereby increase a compressive modulus of the ECM hydrogel by at least 10%.

13. The method of claim 12, wherein said reinforcing agent is a polyaldehyde.

14. The method of claim 12, wherein said reinforcing agent is an oxidized, poly-aldehyde saccharide.

15. The method of claim 12, wherein said contacting is with a culturing medium that comprises said reinforcing agent.

16. A cellularized, engineered retinal construct generated according to the method of claim 1.

17. A cellularized engineered retinal construct comprising endothelial cells and retinal cells distributed within a chemically cross-linked ECM hydrogel, wherein said ECM hydrogel is chemically cross-linked by a biocompatible small-molecule reinforcing agent that is capable of chemically interacting with the ECM hydrogel under conditions that maintain viability of the cells, and wherein a compressive modulus of the ECM hydrogel is higher by at least 50% than a compressive modulus of the ECM hydrogel which is not chemically cross-linked.

18. A method of treating a disease or condition associated with a damaged retina in a subject in need thereof, the method comprising implanting the cellularized construct of claim 1 into the subject, thereby treating the condition.
